(19) United States
(12) Patent Application Publication
(10) Pub. No.: US 2010/0274218 A1
(43) Pub. Date: Oct. 28, 2010

(54) COMMUNICATIONS FOR MEDICINAL FLUID DELIVERY SYSTEM

(75) Inventors: Ofer Yodfat, Maccabim-Reut (IL); Avihoo P. Keret, Kfar Vradim (IL); Tsabar Mor, St. Naharia (IL)

Correspondence Address: MINTZ LEVIN COHN FERRIS GLOVSKY & POPEO, ONE FINANCIAL CENTER, BOSTON, MA, 02111, US

(73) Assignee: MEDINGO LTD, YOQNEAM ILLIT (IL)

(21) Appl. No.: 12/665,995

(22) PCT Filed: Jun. 22, 2008
(86) PCT No.: PCT/IL08/00842

§ 371 Date: Apr. 7, 2010

Related U.S. Application Data
(60) Provisional application No. 60/936,726, filed on Jun. 22, 2007.

(51) Int. Cl. A61M 37/00; H04L 9/32; G06F 15/16; H03M 13/03; G06F 11/10; H04B 15/00

(52) U.S. Cl. 604/504; 713/168; 709/247; 714/795; 709/206; 604/503; 714/E11.032; 714/746; 455/63.1

(57) ABSTRACT

Disclosed are methods, devices, systems and computer program products for communicating between at least one unit of a therapeutic fluid dispensing device and at least another unit of the dispensing device. The method includes performing a determination whether a first frequency can be used to transmit a message related to operations of the dispensing device, and transmitting the message based on the determination.

### CROSS-REFERENCE TO RELATED APPLICATIONS

This application claims priority to provisional U.S. application Ser. No. 60/936,726, entitled “COMMUNICATIONS FOR MEDICINAL FLUID DELIVERY SYSTEM”, and filed Jun. 22, 2007, the content of which is hereby incorporated by reference in its entirety.

### FIELD

The present disclosure generally relates to messaging, and, in particular, messaging among one or more of an infusion device, a remote control unit, and a sensor.

### BACKGROUND

Medical treatment of several illnesses requires continuous drug infusion into various body compartments using, for example, subcutaneous and intra-venous injections. For example, diabetes mellitus patients require the administration of varying amounts of insulin throughout the day to control their blood glucose levels. In recent years, ambulatory portable insulin infusion pumps have emerged as a superior alternative to multiple daily injections of insulin by syringe. These pumps, which deliver insulin at continuous basal rates as well as in bolus volumes, were developed to liberate patients from repeated, self-administered injections, and allow them to maintain a near-normal daily routine. Another example is post surgery pain that requires relief by medication (e.g., opium derivatives). These drugs can be locally delivered to the subcutaneous tissue surrounding the incision scar avoiding systemic side effects of oral or intravenous analgesics. Another example is cancer patients that require continuous delivery of chemotherapy medications via an open vein access port.

Modern drug delivery devices and medical sensors demand a great deal of information relating to parameters and conditions of the treatment and medical state of the patient. Such information typically includes characteristics of the drug dosage and data relating to the conditions of the patient's body. The information may be used to provide improved patient treatment, to enable self-monitoring by the patient, and to improve the operation of the drug delivery device.

The first generation of portable insulin pump was a “pager-like” device attached to a patient's belt. The first generation device included a reservoir within the device housing. A long tube delivered insulin from the pump attached to a patient's belt to a remote insertion site. Example of such first generation devices are described, for example, in U.S. Pat. Nos. 3,631,847, 3,771,694, 4,657,486 and 4,544,369, the contents of all of which are hereby incorporated by reference in their entireties. These first generation devices included a control panel combined with the pump—resulting in relatively large dimensions. Although bulky, large, and heavy, these first generation devices provided a significant improvement over multiple daily injections. The first generation devices were uncomfortable, bulky devices with a long tube. Consequently, these first generation devices were rejected by the majority of diabetic insulin users because the devices impacted regular activities, such as sports and swimming.

To avoid the tubing limitations, a second-generation portable insulin pump was proposed. The second-generation portable insulin pump included a remote controlled device adhered to the skin (“remote skin adherable device”). The remote skin adherable device included a housing having a bottom surface adapted for contact with the patient's skin, a reservoir contained within the housing, and an injection needle adapted for fluid communication with the reservoir. These remote skin adherable devices had a separate control unit, thus resulting in smaller infusion pumps. This second generation paradigm is described, for example, in pending U.S. patent application Ser. No. 11/397,115, filed on Apr. 3, 2006, entitled “Systems and methods for sustained medical infusion and devices related thereto”, the content of which is hereby incorporated by reference in its entirety.

In the above-referenced U.S. patent application (the '115 application) an infusion device was proposed that included a remote controlled skin adherable dispensing patch including a reusable part and a disposable part. Moreover, in that infusion device, the control panel is separated from the dispensing unit, requiring some form of transmission or communication mechanism.

Adherable devices with a remote control give rise to new problems. First, the transmission between the remote control and the adherable device may be corrupted or disrupted. Another drawback is that there are only a limited number of frequencies open for transmissions, such as the Industrial, Scientific, and Medical (ISM) radio band. However, the ISM radio band includes many other wireless devices, such as wireless devices equipped with WiFi (described by The Institute of Electrical and Electronics Engineers (IEEE) 802.11 standard) and wireless devices equipped with Bluetooth (developed by the Bluetooth Special Interest Group (SIG) and covered by the IEEE 802.15 standard). These wireless protocols are used by many devices, such as cordless phones, cellular phones and their accessories, personal computers, hand held computers, and the like. There also other devices, such as microwave ovens, switches, and electric motors, that cause radio frequency interference (RFI) in the ISM radio band. As wireless and miniaturization technologies improve, electrical devices' reliance on wireless as a mechanism for communications will continue to rise, thus exacerbating interference problems (e.g., causing jamming and noise problems). The multiplicity of communications and messages on the same frequency may also give rise to problems related to false messages from other devices mistakenly interpreted as a legitimate message.

Communication between a remote control unit and infusion device are known. Communications problems and programming of infusion pump were known especially in implantable devices. Implantable infusion pumps for infusion of, e.g., insulin, are described, for example, in U.S. Pat. No. 4,494,950 and in the publication by W. Schubert et al., “An implantable artificial pancreas,” Medical and Biological Engineering & Computing, 1980, 18, pp. 527-537, the contents of which are hereby incorporated by reference in their entireties. In the latter document, an artificial implantable pancreas is described in which in a first mode of operation, a glucose sensor transmits the actual blood glucose level to a control unit, in which the amount of insulin to be infused is calculated on the basis of patient specific parameters, recorded in a program memory, and corresponding control signals for a dosing unit are determined by a control procedure. If no sensor is used or if the sensor employed fails, the dosing unit is controlled in a second mode of operation by a stored dosing program. Thus, the first mode of operation corresponds to a closed control loop and the second mode of operation corresponds to an open control loop.

In U.S. Pat. No. 5,558,640, the content of which is hereby incorporated by reference in its entirety, a system is described having a programmable implantable infusion pump with means for bi-directional telemetry communication and a programmable implantable physiological sensor for controlling an implantable device in response to changes in the parameter sensed by the sensor. The controller of these devices comprises programs for scheduling of the dosing unit (i.e., the implantable infusion pump). In this system, statistical calculations are preformed to predict the infusion schedule and to store a patient's dosing plan.

Continuous glucose monitors are described, for example, in U.S. Pat. Nos. 5,390,671 and 6,143,164, the contents of which are hereby incorporated by reference in their entireties. These devices monitor glucose levels in the subcutaneous compartment.

In pending U.S. patent application Ser. Nos. 11/706,606 and U.S. Provisional Application Nos. 60/842,869 and 60/848,511, the contents of all of which are hereby incorporated by reference in their entireties, a dual function dispensing unit is described that can dispense fluids and continuously monitor body analyte (i.e., insulin dispensing and glucose monitoring). Those applications describe a closed loop system for insulin delivery according to glucose levels. The dual-function dispensing unit includes a reusable part and a disposable part. The dual-function dispensing unit may include a remote control.

The communications problems and error tolerance of transmissions between a remote control unit and a medical device are described, for example, in U.S. Pat. Nos. 7,024,245; 6,564,105; and 7,171,274, and in PCT Publication No. 2006/133851, the contents of all of which are hereby incorporated by reference in their entireties.

A problem that was not resolved by stand-alone infusion pumps is monitoring any irregular activities of a user, such as when a treatment is missed or skipped by a user. This is may be solved using a closed loop system, such as the closed loop system described in U.S. application Ser. No. 11/706,606, assigned to Medingo LTD, filed Feb. 14, 2007, and entitled “System and methods for sensing analyte and dispensing therapeutic fluid,” the content of which are hereby incorporated by reference in its entirety. Even though there are some medical conditions that can be measured, such as pain, there are other circumstances that might result in mistreatment, such as being preoccupied, vision related mistakes, and other errors related to poor physical condition.

The ISM radio band may be used by many devices transmitting at these frequencies. On the other hand, there are major improvements in central processing unit (CPU) power and data storage elements, resulting in higher capacity and lower price integrated circuits (IC) with improved calculation abilities. Consequently, these developments may be used to provide secure and reliable communications and message transfer for infusion pumps and other portable medical devices at lower cost.

### SUMMARY

In some embodiments of the present disclosure, reliable communications between the remote control and the infusion pump secured to the patient are provided. Such reliable communications by a portable device may be used to support continuous monitoring of glucose. The continuous monitoring device can be incorporated within the dispensing device, thus having both sensing and dispensing capabilities.

In some embodiments of the present disclosure, an error correction module for the correction of interrupted (e.g., corrupted) messages sent to, or received from, an infusion pump, a sensor for measuring analyte (i.e., glucose) level in the patient body, or a remote control unit for the sensor (or the infusion pump) are provided. The infusion pump or the sensor may include two parts, a disposable part and a reusable part. After connecting the reusable and disposable parts, the whole device has a thin profile and relatively small footprint—making the device discreet.

In some embodiments, validating messages processed by an infusion pump, a sensor for measuring analyte (i.e., glucose) level in the patient body, or a remote control unit of these devices may be implemented.

In some embodiments of the present disclosure, an advanced data processing feature is provided that is capable of compressing data, encrypting data, correcting transmission errors, and providing some advanced features (e.g., prediction, learning, or decision making capabilities) to the infusion pump, a sensor for measuring analyte (i.e. glucose) level in the patient body, and/or the remote control unit of these devices to enable alarms when there is a communication failure.

Accordingly, the present disclosure describes various embodiments of methods, apparatuses and systems, including (for example) computer program products, for messaging. In some embodiments, an external, miniature, portable, programmable fluid dispensing unit configured to communicate with a remote control that contains advanced processing ability is provided. In some embodiments, there is provided a small, low cost, portable dispensing unit, including disposable and reusable parts is provided. The processing ability may be implemented in one or more parts of the system and/or by providing a telemetry module, e.g., a wireless mechanism between the dispensing unit and the remote control.

In some embodiments, there is provided a method for communicating between a remote control unit and at least one of a dispensing unit for delivering medicinal fluids and/or a sensor for measuring glucose level. The method may include compressing a message to reduce the length of the message, the message including data representative of at least one of patient information, a level of blood glucose, and a dosage. The message may be encrypted using a key enabling the message to be recognized by at least one of the remote control unit, the dispensing unit and the sensor. The message may be transmitted using a wireless protocol.

Variations may include one or more of the following features. An available frequency may be determined for transmitting the message. A second frequency may be determined for transmission of the message, when a first frequency is not available for the transmission. The message may be transmitted wirelessly using a spread spectrum protocol from one of the remote control, the dispensing unit, and the sensor to one of the remote control, the dispensing unit, and the sensor. The message may be interleaved before transmission. The message may be coded for error correction before transmission. The message may be received. A similarity check may be performed on the message using stored information representative of whether the message is valid.

In some embodiments, the data produced by any component of the device (e.g., the dispensing unit with remote control unit, sensor unit, sensor with remote control, and integrated dispensing pump with sensor, and remote control unit) may be encrypted, compressed, and stored. The stored, encrypted, and compressed message may then be transmitted from any unit of the device to any other unit of the device. The received message is then decompressed, decrypted, and the logical context of the message is checked to determine if the received message is valid. If the message can be decrypted correctly and its content is logical, then the message is executed or otherwise acted upon. Messages may be stored on at least one of the parts (e.g., the dispensing unit, sensor unit, integrated dispensing unit with sensor, and remote control unit) receiving the message and one of the parts transmitting the message. In some implementations, when storing messages, at least one of the receiving parts and transmitting parts may optimize usage of the device by the learning feature described below.

In some embodiments, a message may be encrypted to increase the security of communications between the remote control unit and the dispensing unit. Secure communications may facilitate providing a broad and comprehensive database of patient conditions and treatment information—resulting in improved treatment for a patient. In some implementations, in order to store as much data as possible regarding a patient, the data (e.g., of a message) is compressed before storage. In some implementations, the logical examination of data (e.g., commands and input values) may increase the assurance and reliability of the dispensing unit. Moreover, as corrupted transmissions may lead to a communication breakdown (e.g., a failure), it may be beneficial to notify the user when a communication breakdown occurs and avoid and remedy such breakdowns. Therefore, it may be useful to use reliable transmission methods (e.g., frequency hopping, orthogonal frequency division multiplexing (OFDM), and spread spectrum transmission) capable of operating in the presence of interference (e.g., noise, jamming, co-channel interference, and other signals).

In some embodiments, there is provided a fluid delivery device that contains one or more processing capabilities to produce encrypted data.

In some embodiments, there is provided a fluid delivery device that contains one or more processing capabilities to produce compressed data.

In some embodiments, there is provided a fluid delivery device that contains one or more processing capabilities for decompressing data.

In some embodiments, there is provided a fluid delivery device that contains one or more processing capabilities for decrypting data.

In some embodiments, there is provided a fluid delivery device that contains one or more storage devices for storing compressed data.

In some embodiments, there is provided a fluid delivery device that contains one or more storage devices for storing encrypted data.

In some embodiments, there is provided a fluid delivery device that contains one or more telemetry components for transmitting or receiving messages (including data and/or commands).

In some embodiments, there is provided a fluid delivery device that contains one or more telemetry components for transmitting or receiving compressed data using frequency hopping.

In some embodiments, there is provided a fluid delivery device that contains one or more telemetry components for transmitting or receiving encrypted data using frequency hopping.

In some embodiments, there is provided a fluid delivery device that contains one or more logical data processing ability.

In some embodiments, there is provided a fluid delivery device that contains one or more logical data processing capabilities for decision-making, such as learning and prediction features described below.

In some embodiments, there is provided a dispensing unit with a remote control that contains one or more machine learning abilities.

In some embodiments, there is provided a dispensing unit with a remote control that contains one or more statistical analysis features.

In some embodiments, there is provided a dispensing unit with a remote control that contains one or more error corrections features. The phrase “dispensing patch” also refers to one or more of the following: an infusion device; dispensing pump, dispensing unit, dispensing patch unit, an infusion device with remote control; an infusion device with a reusable part and disposable part; a sensor with a remote control; a sensor with an infusion pump (e.g., implemented as two separated devices); a sensor integrated with an infusion pump; a sensor integrated with an infusion pump comprising a reusable part and disposable part; a sensor integrated with an infusion pump and a remote control; and a sensor integrated with an infusion pump and a remote control comprising a reusable part and disposable part.

In one aspect, a method for communicating between at least one unit of a therapeutic fluid dispensing device and at least another unit of the dispensing device is disclosed. The method includes performing a determination whether a first frequency can be used to transmit a message related to operations of the dispensing device, and transmitting the message based on the determination.

Embodiments of the method may include one or more of the following features.

The message related to the operations of the dispensing device may include data relating to operation of one or more of, for example, the at least one unit of the therapeutic fluid dispensing device and/or the at least another unit of therapeutic fluid dispensing device.

Performing the determination whether the first frequency can be used to transmit the message may include performing a comparison of detected radio frequency interference (RFI) associated with the first frequency to an associated RFI threshold. Transmitting the message based on the determination may include transmitting the message using the first frequency if the detected RFI is below the associated RFI threshold. Transmitting the message based on the determination may include transmitting the message using a second frequency if the detected RFI is above the associated RFI threshold.

The RFI threshold may be adjustable according to one or more of, for example, time elapsed from processing the message and transmitting it, message type, number of failed transmissions, number of corrupted messages, time interval in which the detected RFI is above the associated RFI threshold level, transmission power, bandwidth, modulation technique and/or the bit rate of the transmission.

The method may further include determining a second frequency to transmit the message in response to the determination being that the first frequency cannot be used to transmit the message.

The method may further include generating one or more user-notifications regarding operations of the dispensing device. Generating the one or more user-notifications may include generating one or more of, for example, a visual notification, an audio notification and/or a user-readable notification. Generating the one or more user-notifications may include generating a user-notification in response to the determination being that the first frequency cannot be used to transmit the message.

The method may further include encrypting at least one portion of the message. Encrypting the at least one portion of the message may include using at least one transmitting encryption key, the transmitting encryption key enabling the message to be recognized by one or more of, for example, a remote control unit of the dispensing device, a dispensing unit to deliver the therapeutic fluids to a patient and a sensor to measure bodily analyte level of the patient. The transmitting encryption key may include a variable encryption key. The transmitting encryption key comprises a fixed encryption key.

The method may further include receiving the transmitted message, and decrypting the received message using at least one receiving encryption key. The at least one receiving encryption key may include one of the at least one transmitting encryption key. The at least one receiving encryption key may include a receiving encryption key different from the at least one transmitting encryption key.

The method may further include compressing at least one portion of the message to reduce message length. The method may further include encrypting the at least part of the message. Compressing the at least one portion of the message may include compressing the at least one portion of the message at one of, for example, a remote control unit of the dispensing device, a dispensing unit to deliver the therapeutic fluids to a patient and a sensor to measure bodily analyte level of the patient.

Transmitting the message may include transmitting the message to at least one of, for example, a remote control unit of the dispensing device, a dispensing unit to deliver the therapeutic fluids to a patient and/or a sensor to measure bodily analyte level of the patient.

The message related to operations of the dispensing device may include data representative of information regarding at least one of, for example, a patient, a level of bodily analyte of the patient and/or a dosage of therapeutic fluid to be dispensed to the patient.

The method may further include transmitting the message wirelessly using a spread spectrum protocol from the at least one unit of the dispensing device to the other unit of the dispensing device.

The method may further include interleaving the message before transmitting the message.

The method may further include coding the message to enable error correction of the message. Coding the message to enable error correction of the message may include coding the message using Viterbi-related error-correction coding schemes.

The method may further include receiving the transmitted message. The method may further include performing a similarity check to determine whether the received message is valid based, at least in part, on stored information representative of message content. The method may further include decoding the message such that at least some missing data and/or errors in the received message are recovered to provide at least some of original content of the transmitted message.

The message related to operations of the dispensing device may include one or more of, for example, an op-code, a time signature, a dosage of therapeutic fluid representative of at least one of a concentration of a therapeutic fluid or analyte, a weight of the therapeutic fluid, an active units of the therapeutic fluid, a pump stroke of the therapeutic fluid, an amount of analyte, a glucose level, information regarding a condition of the patient, patient information representative of at least one of an age, a weight, a blood pressure, a heart rate, program code, control information or signals, a frame synchronization mechanism, an error detection code, a telemetry identifier and/or a clock synchronization.

Transmitting the message may include transmitting at least one portion of the message using a first selected transmission frequency and at least another portion of the message using a second selected transmission frequency.

Transmitting the message may include storing, according to a first ordering pattern, portions of the message into a buffer, retrieving, according to a second ordering pattern, the stored portions of the message, and transmitting the portions of the message retrieved according to the second pattern in the order in which the portions were retrieved. Storing, according to a first ordering pattern, portions of the message may include storing the portions of the message in the buffer according to a row-by-row pattern. Retrieving, according to the second ordering patterns, the stored portions of the message, may include retrieving the portions of the message from the buffer according to a column-by-column pattern.

Transmitting the message may include transmitting the message using a wireless communications protocol based on at least one of, for example, Wireless Application Protocol (WAP), WiFi and/or WiMax.

Transmitting the message may include transmitting the message using a wireless communications protocol based on Low Power Bluetooth protocol.

Transmitting the message may include transmitting the message in the Industrial, Scientific and Medical (ISM) radio bands. Transmitting the message in the ISM radio bands may include transmitting the message in a radio band corresponding to frequency of between 2.4 to 2.5 Giga Hertz.

In another aspect, a method for communicating a message between at least one unit of a therapeutic fluid dispensing device and at least another unit of the dispensing device is disclosed. The method includes transmitting at least a portion of a message relating to operations of the therapeutic fluid dispensing device using a first frequency, and transmitting at least another portion of the message using a second frequency. The second frequency is different than the first frequency.

Embodiments of this method may include any of the features described above in relation to the first described method.

In a further aspect, a device to deliver therapeutic fluid to a patient's body is disclosed. The device includes a dispensing unit to dispense the therapeutic fluid to the patient's body, and a remote control unit to communicate with at least the dispensing unit. At least one of the dispensing unit and the remote control unit is configured to perform a determination whether a first frequency can be used to transmit a message related to operations of the dispensing device, and transmit the message based on the determination.

Embodiments of the device may include any of the features described above in relation to any one of the methods, as well as one or more of the following features.

The at least one of the dispensing unit and the remote control unit may further be configured to encrypt at least one portion of the message. The at least one of the dispensing unit and the remote control unit configured to encrypt the at least one portion of the message may be configured to whiten the message.

The at least one of the dispensing unit and the remote control may be configured to add to at least part of the message forward error correction codes, each forward correction code added to the message based, at least in part, on the content of a part of the message preceding the added code and the content of a part of the message succeeding the added code.

The at least one of the dispensing unit and the remote control unit configured to transmit the message may be configured to store, according to a first ordering pattern, portions of the message into at least one buffer, retrieve, according to a second ordering pattern, the stored portions of the message, and transmit the portions of the message retrieved according to the second pattern in the order in which the portions were retrieved. The at least one buffer may include at least two buffers configured to be accessed separately so that while one of the at least two buffers is accessed another of the at least two buffers can be separately accessed. The at least one of the dispensing unit and the remote control unit configured to store, according to a first ordering pattern, portions of the message, may be configured to store the portions of the message in the buffer according to a row-by-row pattern. The at least one of the dispensing unit and the remote control unit configured to retrieve, according to the second ordering patterns, the stored portions of the message, may be configured to retrieve the portions of the message from the buffer according to a column-by-column pattern.

The at least one of the dispensing unit and the remote control unit may include a communications mechanism to communicate messages, and a processor configured to process the message to be communicated.

The device may further include a sensor to measure bodily analyte level in the patient's body. The sensor may include a communications mechanism to communicate with at least one of the remote control unit and the dispensing unit, and a processor configured to process transmissions to be communicated.

The at least one of the dispensing unit and the remote control unit may further be configured to whiten the message, add to the message forward error correction codes, each forward correction code added to the message based, at least in part, on the content of a part of the message preceding the added code and the content of a part of the message succeeding the added code, store the message in at least one buffer, and transmit at least one portion corresponding to the message using a first transmission frequency and at least another portion corresponding to the message using a second transmission frequency.

The at least one of the dispensing unit and the remote control unit may further be configured to whiten the message, add to the message forward error correction codes, each forward correction code added to the message being generated based, at least in part, on the content of a part of the message preceding the added code and the content of a part of the message succeeding the added code, store the message in at least one buffer, and transmit the message wirelessly using a spread spectrum protocol to at least another unit of the device.

The at least one of the dispensing unit and the remote control unit may further be configured to whiten the message, store the message in at least one buffer, and transmit at least one associated portion of the message using a first selected transmission frequency and at least another associated portion of the message using a second selected transmission frequency.

The at least one of the dispensing unit and the remote control unit may further be configured to whiten the message, code the message to enable error correction of the message using Viterbi-based techniques, and store the message in at least one buffer.

The at least one of the dispensing unit and the remote control unit may further be configured to encrypt the message, code the message to enable error correction of the messages, and store the messages in at least one buffer.

In yet another aspect, disclosed is a computer program product to communicate between at least one unit of a therapeutic fluid dispensing device and at least another unit of the therapeutic fluid dispensing device, the computer program product comprising computer instructions stored on a computer readable medium. The computer instructions, when executed, cause a processor-based device to perform a determination whether a first frequency can be used to transmit a message related to operations of the dispensing device, and based on the determination, provide the message to a transmitter to wirelessly transmit the message to the at least other unit of dispensing device.

Embodiments of the computer program product may include any of the features described above in relation to any one of the methods and device.

In another aspect, a system for communicating between a remote control unit and at least one of a dispensing unit to deliver therapeutic fluids to a body of a patient and a sensor to measure bodily analyte level of the patient is disclosed. The system includes one or more processors to perform a determination whether a first frequency can be used to transmit one or more messages related to operations of at least one of the remote control, the dispensing unit and the sensor, and one or more transmitters to transmit the one or more messages from at least one of the remote control unit, the dispensing unit, and the sensor to at least another of the remote control unit, the dispensing unit, and the sensor.

Embodiments of the system may include any of the features described above in relation to any one of the methods, device and computer program product.

In an additional aspect, a method for communicating between a remote control unit and at least one of a dispensing unit for delivering therapeutic fluids to a body of a patient and a sensor for measuring bodily analyte level is disclosed. The method includes encrypting at least one portion of a message related to operations of one or more of the remote control unit, the dispensing unit, and the sensor, and transmitting the message from at least one of the remote control unit, the dispensing unit, and the sensor to at least another of the remote control unit, the dispensing unit, and the sensor.

Embodiments of the method may include any of the features described above in relation to any one of the methods, device, computer program product and system, as well as any one or more of the following features.

Encrypting the at least one portion of the message may include using at least one transmitting encryption key, the transmitting encryption key enabling the message to be recognized by one or more of, for example, the remote control unit, the dispensing unit and/or the sensor.

The transmitting encryption key may include a variable encryption key.

The transmitting encryption key may include a fixed encryption key.

The method may further include receiving the transmitted message, and decrypting the received message using at least one receiving encryption key. The at least one receiving encryption key may include one of the at least one transmitting encryption key. The at least one receiving encryption key may include a receiving encryption key different from the at least one transmitting encryption key.

Encrypting the at least one portion of the message may include applying asymmetric cryptography to the at least one portion of the message.

Encrypting the at least one portion of the message may include encrypting the at least one portion of the message using a public key, wherein the at least one encrypted portion can be decrypted by a private key of an intended recipient of the message.

Encrypting the at least one portion of the message may include encrypting the at least one portion of the message such that only a decryption key of an intended recipient of the message can be used to decrypt the message to recover original content of the message.

The method may further include performing a determination whether a first frequency can be used to transmit the message. Transmitting the message may include transmitting the message based, at least in part, on the determination.

Performing the determination whether the first frequency can be used to transmit the message may include performing a comparison of detected radio frequency interference (RFI) associated with the first frequency to an associated RFI threshold. Transmitting the message based on the determination may include transmitting the message using the first frequency if the detected RFI is below the associated RFI threshold. Transmitting the message based on the determination may include transmitting the message using a second frequency if the detected RFI is above the associated RFI threshold. The RFI threshold may be adjustable according to one or more of, for example, time elapsed from processing the message and transmitting it, message type, number of failed transmissions, number of corrupted messages, time interval in which the detected RFI is above the associated RFI threshold level, transmission power, bandwidth, modulation technique and/or the bit rate of the transmission.

In a further aspect, a device to deliver therapeutic fluid to a patient's body is disclosed. The device includes a dispensing unit to dispense the therapeutic fluid to the patient's body, and a remote control unit to communicate with at least the dispensing unit. At least one of the dispensing unit and the remote control unit is configured to encrypt at least one portion of a message related to operations of one or more of the remote control unit and the dispensing unit, and transmit the message from one of the remote control unit and the dispensing unit to another of the remote control unit and the dispensing unit.

Embodiments of the device may include any of the features described above in relation to any one of the methods, device, computer program product and system.

In another aspect, disclosed is a computer program product to communicate between at least one unit of a therapeutic fluid dispensing device and at least another unit of the therapeutic fluid dispensing device, the computer program product comprising computer instructions stored on a computer readable medium. The computer instructions, when executed, cause a processor-based device to encrypt at least one portion of a message related to operations of the dispensing device, and provide the message to a transmitter to cause the message to be transmitted from the at least one unit of the dispensing device to the other unit of the dispensing device.

Embodiments of the computer program product may include any of the features described above in relation to any one of the methods, devices, computer program product and system.

In yet a further aspect, a system for communicating between a remote control unit and at least one of a dispensing unit to deliver therapeutic fluids to a body of a patient and a sensor to measure bodily analyte level of the patient is disclosed. The system includes one or more processors to encrypt at least one portion of at least one message related to operations of one or more of the remote control unit, the dispensing unit and the sensor, and one or more transmitters to transmit the at least one message from at least one of the remote control unit, the dispensing unit, and the sensor to at least another of the remote control unit, the dispensing unit, and the sensor.

Embodiments of the system may include any of the features described above in relation to any one of the methods, devices, computer program products and system.

In an additional aspect, a method for communicating between a remote control unit and at least one of a dispensing unit for delivering therapeutic fluids to a body of a patient and a sensor for measuring bodily analyte level is disclosed. The method includes compressing at least one portion of a message related to operations of one or more of the remote control unit, the dispensing unit, and the sensor, and transmitting the message from at least one of the remote control unit, the dispensing unit, and the sensor to at least another of the remote control unit, the dispensing unit, and the sensor.

Embodiments of the method may include any of the features described above in relation to any one of the methods, devices, computer program products and systems, as well as any one of the following features.

Compressing the at least one portion of the message may include compressing the at least one portion of the message at one of, for example, the remote control unit, the dispensing unit and/or the sensor.

The method may further include encrypting at least part of the message.

In a further aspect, a device to deliver therapeutic fluid to a patient's body is disclosed. The device includes a dispensing unit to dispense the therapeutic fluid to the patient's body, and a remote control unit to communicate with at least the dispensing unit. At least one of the dispensing unit and the remote control unit is configured to compress at least one portion of a message related to operations of one or more of the remote control unit and the dispensing unit, and transmit the message from one of the remote control unit and the dispensing unit to another of the remote control unit and the dispensing unit.

Embodiments of the device may include any of the features described above in relation to any one of the methods, devices, computer program products and systems.

In another aspect, disclosed is a computer program product to communicate between at least one unit of a therapeutic fluid dispensing device and at least another unit of the therapeutic fluid dispensing device, the computer program product comprising computer instructions stored on a computer readable medium. The computer instructions, when executed, cause a processor-based device to compress at least one portion of a message related to operations of the dispensing device, and provide the message to a transmitter to cause the message to be transmitted from the at least one unit of the dispensing device to the other unit of the dispensing device.

Embodiments of the computer program product may include any of the features described above in relation to any one of the methods, devices, computer program products and systems.

In yet another aspect, a system for communicating between a remote control unit and at least one of a dispensing unit to deliver therapeutic fluids to a body of a patient and a sensor to measure bodily analyte level of the patient is disclosed. The system includes one or more processors to compress at least one portion of at least one message related to operations of one or more of the remote control unit, the dispensing unit and the sensor, and one or more transmitters to transmit the at least one message from at least one of the remote control unit, the dispensing unit, and the sensor to at least another of the remote control unit, the dispensing unit, and the sensor.

Embodiments of the system may include any of the features described above in relation to any one of the methods, devices, computer program products and systems.

It is to be noted that both the foregoing general description and the following detailed description are exemplary and explanatory only and are not restrictive. Further features and/or variations may be provided in addition to those set forth herein. For example, the implementations described herein may be directed to various combinations and subcombinations of the disclosed features and/or combinations and subcombinations of several further features disclosed below in the detailed description.

### BRIEF DESCRIPTION OF THE DRAWINGS

In the drawings:

FIG. 1a-1c are schematic diagrams of a dispensing device including: a single-part dispensing unit and a sensor unit and a two-part dispensing unit (with and without remote control).

FIGS. 2a-b are respective schematic diagrams of an exemplary remote control, a single part dispensing unit, and a two part dispensing unit, both of the dispensing units being secured to a patient's skin by a cradle.

FIGS. 3a-f are schematic diagrams, including diagrams showing internal components, of an exemplary peristaltic dispensing unit and piston dispensing unit with reusable and disposable parts and a remote control unit.

FIG. 4 is a flow diagram of an exemplary process for storing and for transmitting messages.

FIGS. 5a-c are flowcharts of exemplary processes related to a frequency hopping feature.

FIGS. 6a-b are flow diagrams depicting exemplary procedures of buffering and compressing of a message for transmission using frequency hopping.

FIG. 7a-c are schematic diagrams depicting exemplary procedures of encryption and decryption of a message.

FIG. 8 is a flowchart of an exemplary procedure to process a received message.

FIG. 9 is a flow diagram of exemplary operations of message compression and encryption.

FIGS. 10a-c are flow diagrams and a table depicting exemplary operations of message processing by the receiving part.

FIGS. 11a-b are schematic diagrams of an exemplary device implementing a pairing process and the initiation of unique encryption key between the paired devices.

FIG. 12 is a diagram depicting exemplary logical data processing and prediction operations of a therapeutic dispensing device.

### DETAILED DESCRIPTION

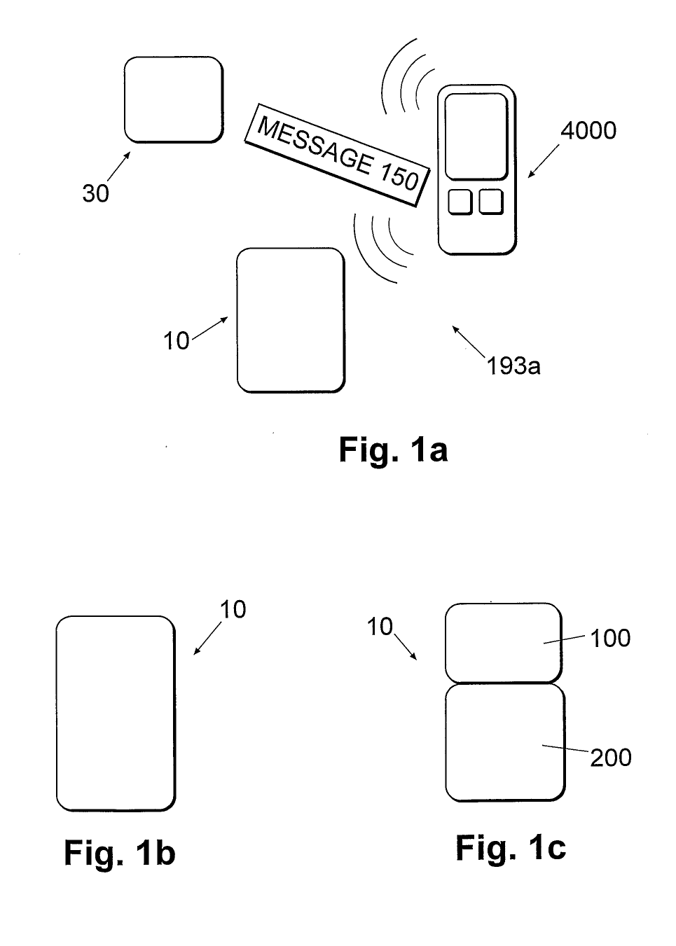

Referring to FIG. 1a, a schematic diagram of an exemplary fluid delivery device 193a is shown. The fluid delivery device includes a dispensing unit 10 which may be adhered (i.e., to stick by, for example, gluing, suction, grasping, or fusing mechanisms) to the patient's body by, for example, attachment to a cradle unit that is glued by medical adhesion and has a gripping mechanism for the dispensing unit as described, for example, in U.S. Provisional Application, No. 60/876,679, filed Dec. 22, 2006, entitled “Improved system, device and method for sustained delivery of a therapeutic fluid”, the content of which is hereby incorporated by reference in its entirety. As further shown, the fluid delivery device also includes a remote control unit 4000 to communicate with the dispensing unit 10 or with sensor unit 30. The dispensing unit 10 may include a single part (as shown in FIG. 1b) or two parts (as shown in FIG. 1c) that include a reusable part 100 and a disposable part 200. The dispensing unit 10 may employ different fluid dispensing mechanisms, such as a syringe-type reservoir with a propelling plunger, a peristaltic positive displacement pump, and the like.

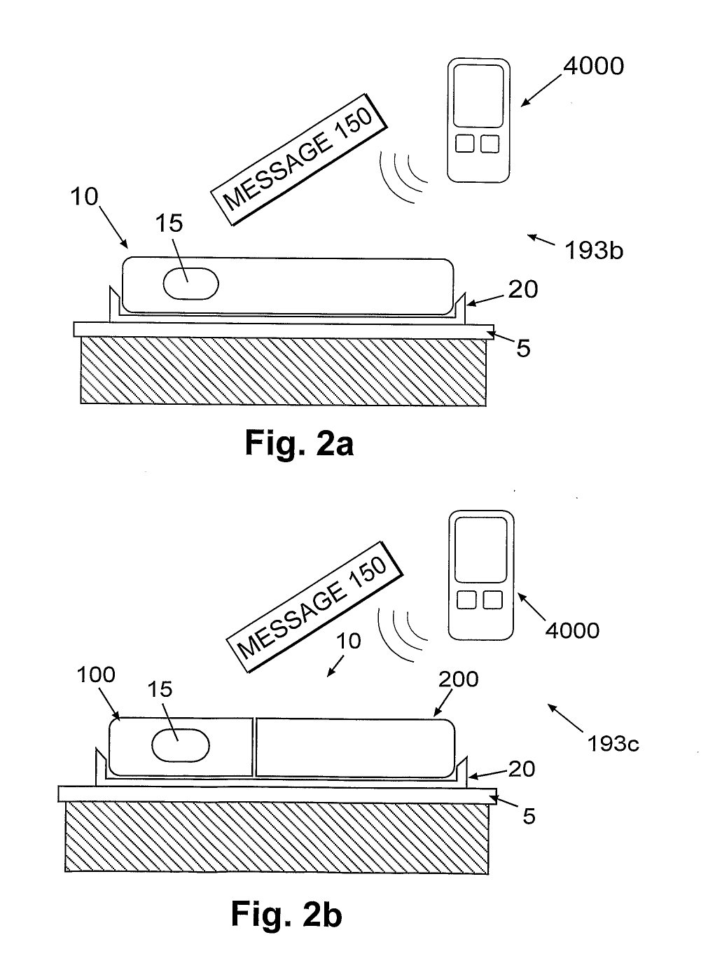

Referring to FIG. 2a, a schematic diagram of a fluid delivery device 193b that includes a single-part dispensing unit 10, a cradle unit 20, and a remote control unit 4000 is shown. In some embodiments, the remote control unit 4000 and the dispensing unit 10 communicate with each other by, for example, transmission of messages (e.g., transmitted wirelessly or through electrical wiring) such as a message 150 depicted in FIG. 2a. The dispensing unit 10 is connected to a cradle unit 20 that is attached to the patient's skin 5. The dispensing unit 10 may be disconnected from or reconnected to the cradle unit 20 by a user, such as a patient using the fluid delivery device. A needle unit comprising a cannula and a penetrating member (not shown) can be inserted through the cradle unit into the body. Fluid delivery operations may be programmed by a remote control unit 4000 or by using a user interface that includes at least one button 15 provided on the dispensing unit 10.

A message communicated by the depicted device, such as message 150, may be related to operations of the device's modules/units, and thus may be related to operation of one or more of, for example, the remote control unit, the dispensing unit and/or the sensor. The message relating to the operations of the device's modules/unit may include such data as: an op-code; a time signature; a dosage of therapeutic fluid representative of at least one of a concentration of a therapeutic fluid or analyte, a weight of the therapeutic fluid, an active units of the therapeutic fluid, a pump stroke of the therapeutic fluid; an amount of analyte; a glucose level; information regarding a condition of the patient; patient information representative of at least one of an age, a weight, a blood pressure, a heart rate; program code; control information or signals; a frame synchronization mechanism; an error detection code; a telemetry identifier; and a clock synchronization.

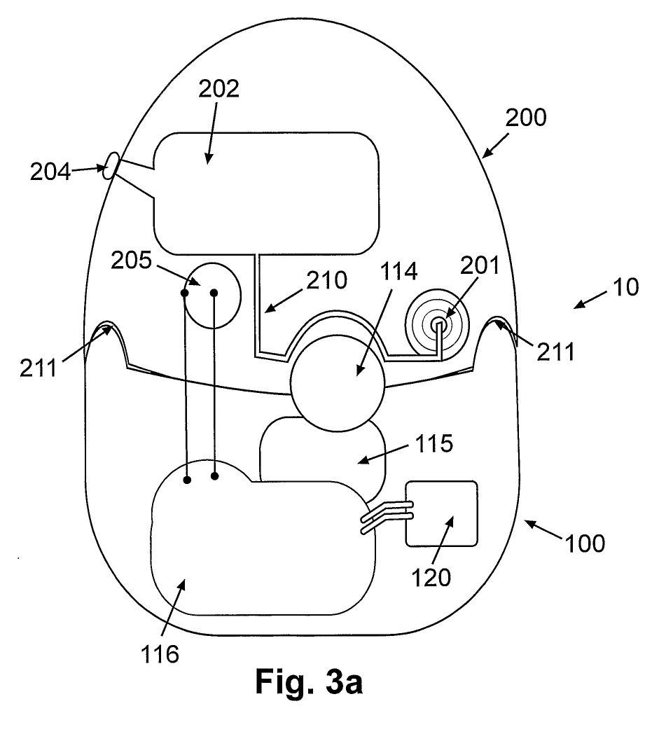

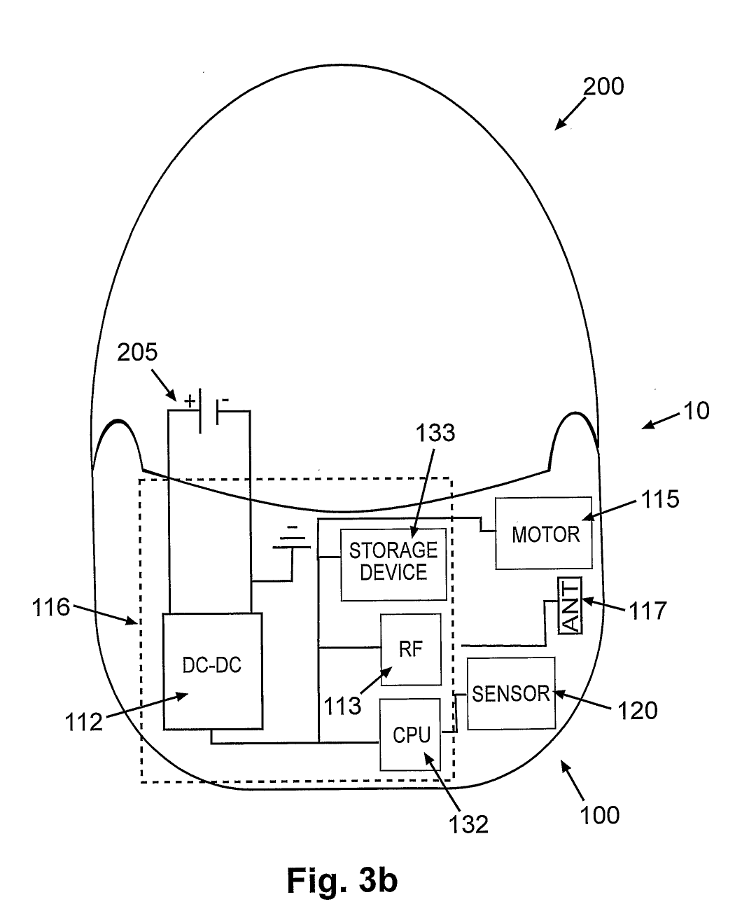

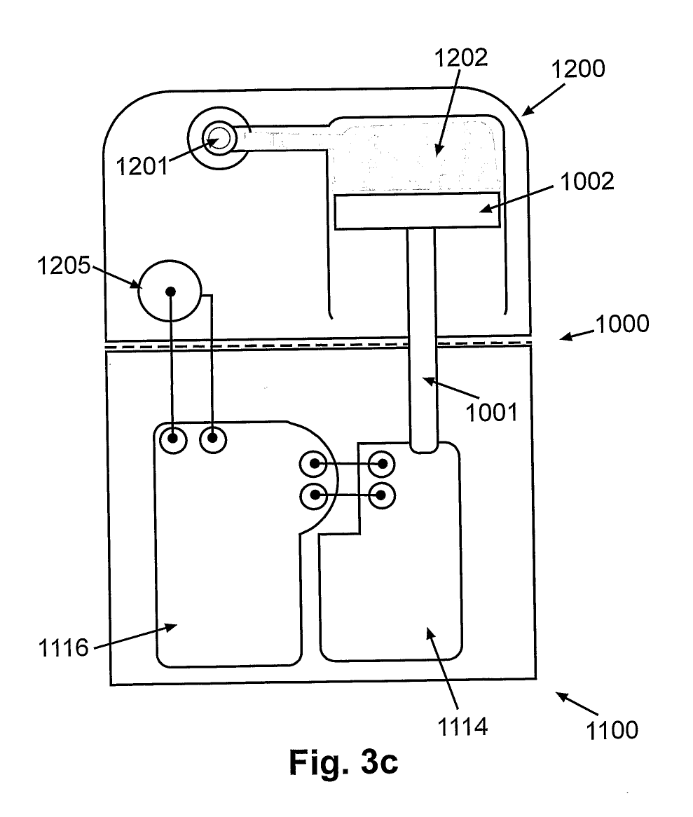

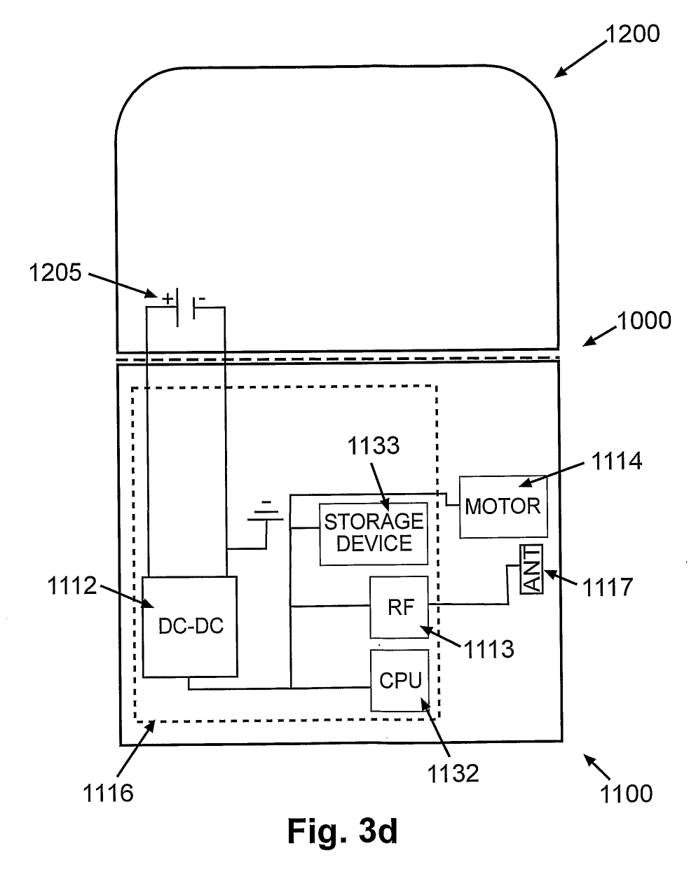

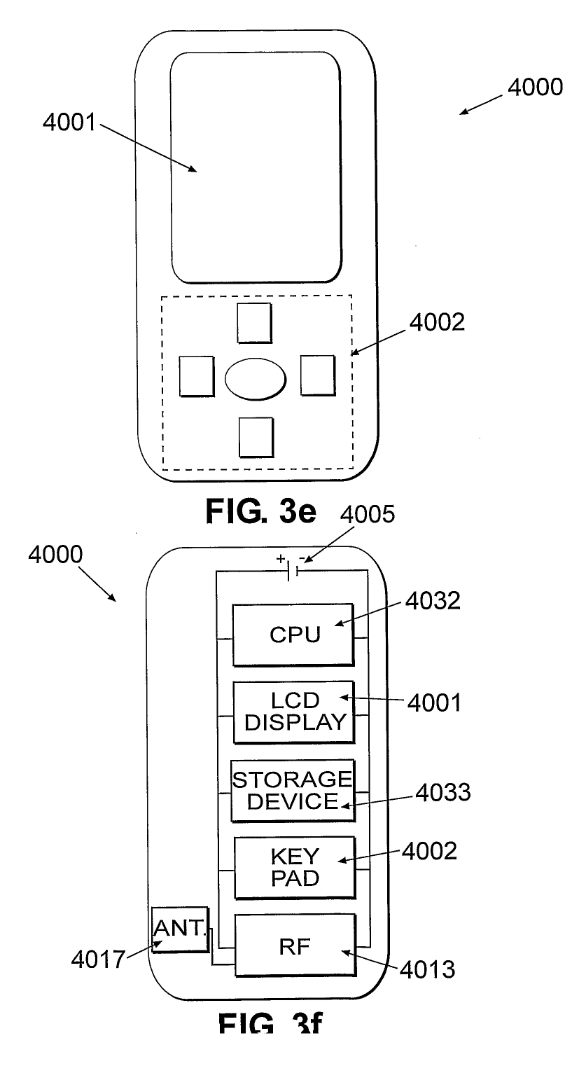

Referring to FIG. 2b, a schematic diagram of an exemplary fluid delivery device 193c comprising a two-part dispensing unit 10, a cradle unit 20, and a remote control unit 4000, is shown. The two-part dispensing unit 10 includes a reusable part 100 and a disposable part 200. One or more buttons 15, constituting, for example, a user interface, may be located on the reusable part 100 of the dispensing unit 10. Exemplary embodiments of the two parts dispensing are further described in FIGS. 3a, 3b, 3c and 3d. Further examples of fluid delivery device implementations that include a dispensing unit are described, for example, in the patent applications assigned to Medingo Medical systems, such as U.S. patent application Ser. No. 11/397,115, filed Apr. 3, 2006, and entitled “Systems and methods for sustained medical infusion and devices related thereto,”, U.S. patent application Ser. No. 11/706,606, filed Feb. 14, 2007, and entitled “System and methods for sensing analyte and dispensing therapeutic fluid,” U.S. Provisional Application No. 60/833,110, filed Jul. 24, 2006, and entitled “Systems, devices and methods for fluid/drug delivery,”, U.S. Provisional Application No. 60/842,869, filed Sep. 6, 2006, and entitled “Fluid delivery system with optical sensing of analyte concentration levels,”, and U.S. Provisional Application No. 60/848,511, filed Sep. 29, 2006, and entitled “Fluid delivery system with electrochemical sensing of analyte concentration levels,”, the contents of all of which are incorporated herein by reference in their entireties. In some implementations, the fluid delivery device may house the relatively more expensive components in the reusable part 100 of the dispensing unit, while housing the relatively less expensive components in the disposable part 200 of the dispensing unit. Consequently, the use of the fluid delivery device of FIG. 2b may be more economical for the manufacturer of the fluid delivery device, the payer (e.g., medical insurance provider) of the fluid delivery device and/or the patient of the fluid delivery device.

Referring to FIG. 3a, a schematic diagram of an exemplary embodiment of the internal arrangement of the dispensing unit 10 is shown. The dispensing unit 10 delivers therapeutic fluid to the user's body using peristaltic pumping mechanism which includes: a rotary wheel 114, a driving mechanism 115 and delivery tube 210. As shown, in some embodiments, the dispensing unit includes a disposable part 200 coupled to a reusable part 100. The disposable part 200 may include one or more of the following: an outlet port 201 for delivering the therapeutic fluid (e.g., medicinal fluid such as insulin) to the patient body; a reservoir for storing therapeutic fluid 202; an inlet port 204 for filling the reservoir with therapeutic fluid; a delivery tube 210 for connecting the reservoir to the outlet port; and a battery 205 to serve as a power source for the electrical components of the infusion pump. The reusable part 100 may include one or more of the following: a driving mechanism 115 including a motor and a gear (not shown); a rotary wheel 114 coupled to the driving mechanism and a part of the delivery tube to provide peristaltic pump operation; a printed circuit board (PCB) 116 with one or more electronic components; and a sensor 120, such as an optic sensor, to measure the glucose level in the patient's body.

Each of the dispensing unit parts e.g., the disposable part 200 and the reusable part 100) may also include a mechanism 211 for coupling the disposable part 200 and the reusable part 100 to each other. In some embodiments, such a coupling mechanism may be implemented as a magnetic-based coupling mechanism, or other types of coupling mechanisms, such as coupling mechanisms that include connectors, clips, clamps, rails, cogs, Velcro, adhesives, etc. An electrical connector may also be used with the mechanical connectors to attach the two parts 100 and 200 while providing an electrical connection to, among other things, power source (e.g., battery) 205. Suitable electrical connectors include commercially available electrical connectors from, for example, Tyco Electronics Corporation.

Referring to FIG. 3b, a schematic diagram of an exemplary implementation of the electrical circuit arrangement 116 of the disposable part 200 and the reusable part 100 when attached together to form a dispensing unit 10 is shown. In some implementations, when the disposable part 200 and the reusable part 100 are attached to each other, the electrical circuit 116 arrangement is powered by electrically connecting the power source 205 to the other components of the electrical circuit arrangement 116.

The electrical circuit 116 may include one or more of the following: a motor driver 115 to drive the pump and a CPU 132 to control and monitor the activity of the pump and of the communications. The CPU 132 may be placed on a printed circuit board (PCB). The electrical circuit 116 may also include a radio frequency (RF) transceiver 113 and an antenna 117 for transmitting and/or receiving transmissions from another device, such as the remote control unit or from another wireless device (e.g. hand held computer, smart phone, and the like). The electrical circuit arrangement 116 also includes a storage device 133 to store messages and other information associated with the functions provided to or by the dispensing and sensing unit. The storage device 133 may be implemented as any type of persistent (non-volatile) storage (e.g., FLASH memory, a hard drive, and the like), although volatile forms of storage may be used as well. Moreover, software-based implementations, for example, database and database management system (DBMS) applications, may be stored on the storage device 133.

Additionally, a sensor 120, such as an optical sensor for determining glucose level in the patient body, may be provided to implement a closed loop or semi closed loop system.

The dispensing unit may also include a DC-DC step-up converter 112 to provide the energy requirements for the electrical components of the electrical circuit arrangement 116.

It is to be noted that FIG. 3b represents an exemplary embodiments of an electrical circuit arrangement, and that other implementations, which may include additional or fewer components compared to the arrangement 116, may be used.

Referring to FIG. 3c, a schematic diagram of another exemplary embodiment of a dispensing unit 1000 is shown. The dispensing unit 1000 of FIG. 3c is configured to deliver therapeutic fluid by a piston infusion pump. Descriptions of dispensing units similar to that shown in FIG. 3c is provided, for example, in co-pending U.S. Provisional Patent Application No. 60/928,815, filed May 11, 2007, entitled “A positive displacement pump,” and assigned to Medingo, the content of which is hereby incorporated by reference in its entirety. In some implementations, the dispensing unit 1000 may include a reusable part 1100 and a disposable part 1200. The dispensing unit 1000 may include one or more of the following: an outlet port 1201 for delivering the therapeutic fluid to the patient body; a reservoir 1202 for storing therapeutic fluid; a power source 1205 that includes, for example, one or more batteries for energizing the electrical components of the infusion pump; a displacement driving mechanism 1114 including a motor and a gear (e.g., the driving mechanism is configured to move a piston, which in some implementations includes a threaded rod 1001 and a plunger 1002); and electronic components 1116, such as a controller, a processor, and/or a transceiver. In the embodiments shown in FIG. 3c, the dispensing unit has no sensing ability, and thus in order to establish a closed loop, the dispensing receives information from another sensing unit or, in some embodiments, the dispensing receives input (e.g., instructions and data) directly from a patient, e.g., via a remote control unit (open loop). It should be noted that any description of communication methods, techniques, procedures and components apply to dispensing unit 10 (shown in FIGS. 1a, 2a and 2b) and also to dispensing unit 1000 of FIGS. 3a and 3b.

Referring to FIG. 3d, a schematic diagram of an exemplary implementation of the electrical circuit arrangement 1116 constituted by the disposable part 1200 and the reusable part 1100 when those two parts are attached to form dispensing unit 1000 is shown

The electrical circuit arrangement 1116 may include one or more of the following: a motor driver 1114; a CPU 1132 that controls and monitors the activity of the pump and of the communications; a RF transceiver 1113; and an antenna 1117 for transmitting and receiving transmissions from another device, such as the remote control unit or from another wireless device (e.g. hand held computer, smart phone, and the like). The electrical circuit arrangement 1116 also includes a storage device 1133 to store messages and other information associated with the functions provided to the dispensing unit. The storage device may be implemented using any type of persistent (non-volatile) storage (e.g., FLASH memory, a hard drive, and the like) and/or less persistent forms (volatile) storage devices. The dispensing unit also includes a DC-DC step up converter 1112 to provide the energy requirements for the electrical components. It is to be noted that FIG. 3d represents an exemplary embodiments of the circuit arrangement, and that in some embodiments additional or fewer components may be used.

Referring to FIGS. 3e and 3f, schematic diagrams of exemplary embodiments of a remote control unit 4000 are shown. The remote control unit 4000, which may be used to control and/or interact with a dispensing unit such as the dispensing unit 10 and/or the dispensing unit 1000 shown in FIGS. 3a-d, includes control buttons 4002, a user interface 4001 (e.g., a liquid crystal display), and a CPU 4032 to control and monitor the communications and other processing performed by the remote control unit.

As shown in FIG. 3f, the remote control unit 4000 includes an RF transceiver 4013 coupled to an antenna 4017 to transmit and/or receive transmissions to and from the dispensing unit, from a sensor, or from another device (e.g. hand held computer, and the like). A storage device 4033 may also be provided to store messages and other information associated with the functions performed by the dispensing and sensing unit, as well as by the remote control. Software-based applications, such as a database, a database management system (DBMS), etc., may be stored on the storage device 4033. These software applications (or computer program product) may be accessed and retrieved by a processing device, such as the CPU 4032, and executed.

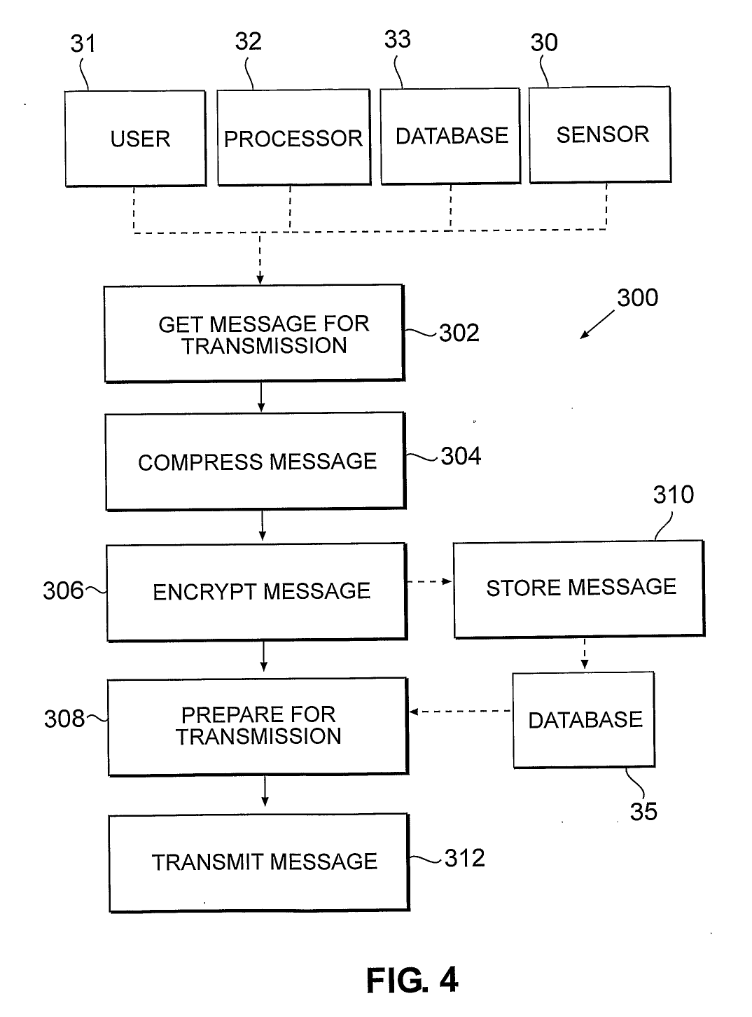

Referring to FIG. 4, a flow diagram of an exemplary process 300 to process and transmit messages is shown. The process 300 may be implemented using one or more portions of the fluid delivery device. For example, messages may be processed for transmission at a sensor unit, a dispensing unit, a remote control unit, or any portion of the fluid delivery device or at an associated accessory including a wireless computer, a hand held computer, a smart phone, a personal digital assistance, and the like.

As shown, in some embodiments, a message may be retrieved 302 by a processor included, for example, within fluid delivery unit 10. The message may be retrieved from (or provided by) one or more of the following: received from a user 31 using a user interface; generated at a processor 32; stored in a database 33 (e.g., such as a message generated at a specific time); retrieved as a result of another incoming message, such as from a sensor 34; or retrieved from any other source.

The data of the retrieved message is compressed 304. The compression may be implemented using any data compression technique. Suitable compression techniques include one or more of the following: a VLC (variable-length code) technique, a Karhunen-Loeve transform, MPEG, 7z, any type of lossy compression technique, any type of lossless compression technique, code book compression techniques, CCITT V.42bis, Lempel-Ziv-Welch (LZW), Huffman coding, Shannon-Fano coding and/or other statistical redundancy procedures. The compression of messages may result in a shorter transmission—making it less likely that a message is corrupted (or interrupted).

The data of the retrieved message is then encrypted 306. The encryption may be implemented using one or more encryption procedures or protocols, such as, for example, SSL (Secure Socket Layer), modified SSL, RSA, SHA-1 (available from Aaben Kryptografi LTD), whitening and International Data Encryption Algorithm (IDEA™) (available from MediaCrypt). Whitening is a symmetric key block cipher, which typically operates on fixed-length groups of bits (i.e. blocks), with an unvarying transformation. When encrypting, a block cipher might take a (for example) 64-bit block of data as input, and output a corresponding 64-bit block of ciphered data. The exact transformation is controlled using a second input—the encryption key. Decryption is similar: the decryption algorithm takes, in this example, a 64-bit block of ciphered data together with the encryption key (now used as decryption key), and yields the original 64-bit block of data.

In some embodiments, the encryption key is unique for any pair of a remote control unit, such as the remote control unit 4000 and a dispensing unit, such as the dispensing unit 10, although a key may be used to encrypt communications for more than a pair of devices. This key may be predetermined or established when the remote control unit 4000 and dispensing unit 10 first establish communication and register with each other (e.g., a so-called “pairing” of remote control unit 4000 and dispensing unit 10). The key may also change from time to time. The use of a unique key may eliminate the need for uniquely identifying the sources and destinations of the messages exchanged between remote control unit 4000 and unit dispensing 10 because the key may only be decrypted by the intended target of the transmission regardless of other devices that may be present. For example, an encrypted message may be transmitted in a radio frequency environment that includes a plurality of devices (e.g., remote control units, dispensing units, sensors, and the like). However, only the intended receiver of that message will be able to successfully decrypt the encrypted message.

In some other embodiments, the encryption key is a hash number of one or more of the communicating devices (e.g., a hash of the serial numbers of remote 4000 and dispensing unit 10). In some embodiments, asymmetric cryptography is applied to at least one portion of the message. In some embodiments, at least one portion of the message may be encrypted such that only a decryption key of an intended recipient of the message can be used to decrypt the message to recover original content of the message. In some embodiments, at least one portion of the message can be encrypted using a public key. The encrypted portion can be decrypted by a private key of an intended recipient of the message. The public key can be embedded in both of the units, as it can be generic key used by all the units constituting the fluid delivery device.

At 308, the message is prepared for transmission over the wireless channel. For example, telemetry equipment (e.g. a modem) may add a preamble and a synchronization frame to one or more of the messages before transmission. However, in some embodiments, the message is stored ready for transmission, in which case there may be no need for further processing.

Optionally, the encrypted message may be stored 310 in a storage device (e.g., database 35) that can store the encrypted message in a logbook or as a database 35. The database 35 may be the same database as database 33 used to generate messages (e.g., message 150). In some embodiments, messages may be stored in compressed form, thus alleviating, to some extent, storage requirements. Stored messages (or the data associated with the messages) may be used for product development, validation, maintenance, and quality assurance of the fluid delivery device. The stored messages (or the data associated with the messages) may also be used to generate a profile for a user or to facilitate other medical applications.

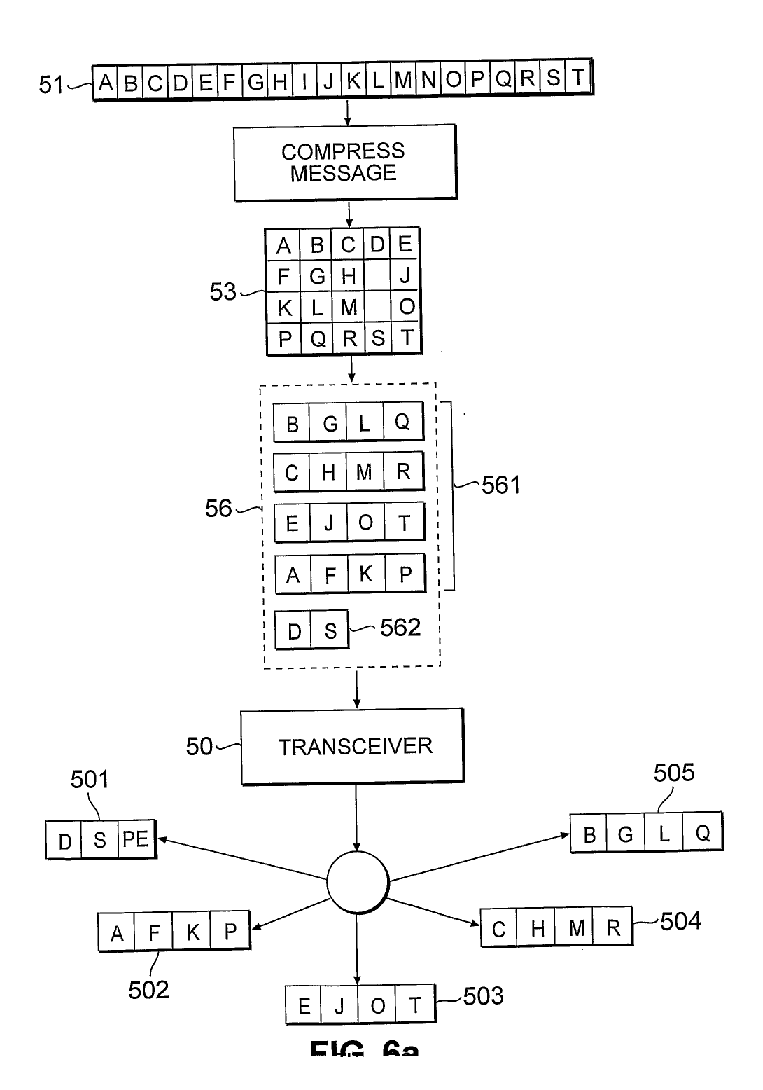

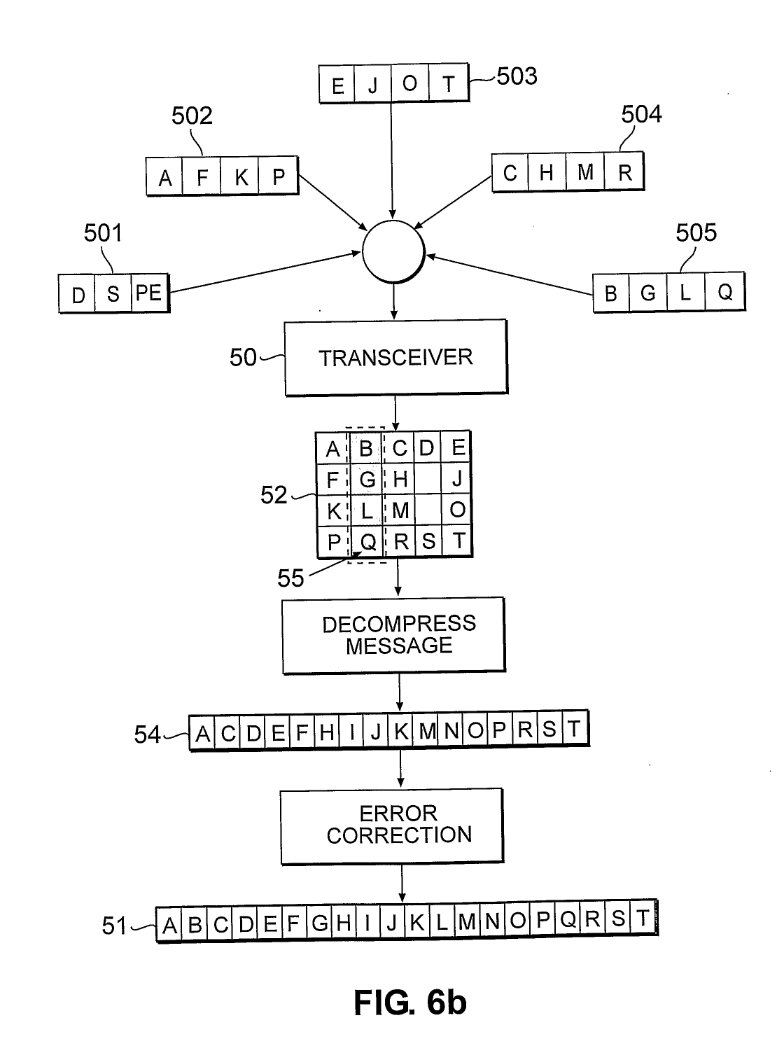

As further shown in FIG. 4, one or more of the encrypted messages are transmitted 312. Referring to FIG. 6a, a flow diagram depicting processing operations performed on a message 51 is shown. The processing operations may performed on the message may include at least one of encryption and compression. For example, the remote control unit 4000 may transmit one or more messages, such as the message 51 or message 150, to the dispensing unit (e.g., dispensing unit 10). In some embodiments, a stand-alone sensor (e.g., sensor unit 30 shown in FIG. 1) for measuring an analyte (e.g., glucose) level in a patient's body may transmit and/or receive messages from remote control unit 4000 or infusion pump (i.e., dispensing unit 10). Transmission of messages (e.g., message 150 or message 51) may also occur between a sensor and a dispensing unit or in any other combination of a sensor unit, a dispensing unit, and a remote control unit. In addition, communications among multiple sensors may also be implemented as described, for example, in U.S. Pat. No. 5,558,640, entitled “System for infusion of medicine into the body of a patient.”

The transmission of the one or more messages 312 (e.g., transmission of the message 150) may be implemented using any wireless protocol, such as the Wireless Application Protocol (WAP), Bluetooth, WiFi, WiMax. Spread spectrum, and other communications mechanisms/techniques may also be applied.

The message, which may be encrypted and/or compressed, may be stored without transmission, or a stored message may be read and subsequently transmitted, e.g., the transmission may occur after the message had been stored for some time at unit 10, remote control unit 4000, or sensor unit 30.

As noted, before a message is transmitted 312, it may be processed for transmission at 308. This processing may include adding information to the message, such as adding a frame synchronization, a preamble, a time stamp (e.g., the current time), or changing a part of the message, such as updating the time of transmission (e.g., when the message was stored before transmission). In some embodiments, a message may include the serial number of a device or some other identifier for the device, such as a hash number.

In some embodiments, a Hash algorithm may be used to provide a hash number for every device. The Hash numbers may be different for every set of input, but they may not be unique. For example, in some embodiments the Hash number may be determined by finding the residue of division in a large prime number, for instance 827. Thus, every 827 serial numbers (of devices) would have a different hash number, but the 828^{th} serial number would have a hash number identical to the first device.

Although the procedure 300 is depicted in the order shown, the operations of the procedure 300 may be performed in other orders. Moreover, in some implementations, one or more aspects of the procedure 300 may be omitted. For example, compression operations at 304 may be omitted from the procedure 300.

In some embodiments, the compression mode may be determined by the devices (e.g., the remote control unit 4000 and/or the dispensing unit 10) during the pairing process or during regular operation of the fluid delivery device. Thus, the various modules of the fluid delivery device (e.g., dispensing unit 10, remote control unit 4000, and the like) may compress data using one or more techniques. In some embodiments, the particular technique used to perform compression may be changed on predetermined basis, such as when the disposable part is being replaced, the reservoir of the therapeutic fluid is empty or full, and when there are communication errors. In some embodiments, the particular compression technique being used may vary on a temporal basis, e.g., changing the used technique periodically, for example, changing the technique, weekly.

In some embodiments, the technique used to encrypt messages may be changed on predetermined basis, such as when the disposable part is being replaced, the reservoir of the therapeutic fluid is empty or full, and when there are communication errors. In some embodiments, the particular encryption technique being used may be changed periodically, e.g., weekly.

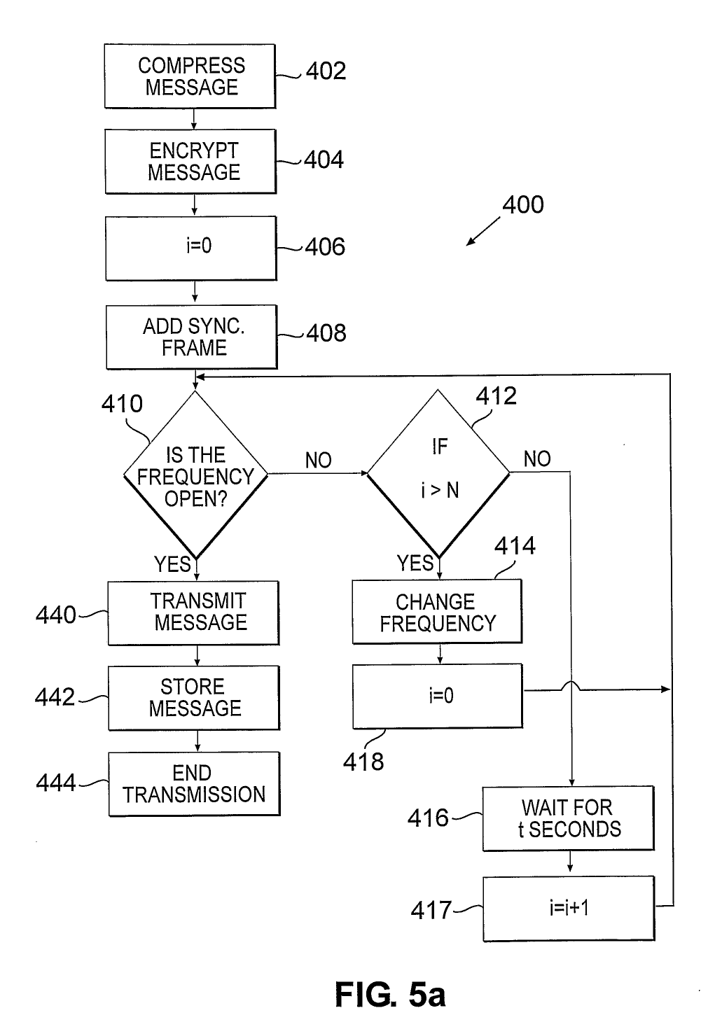

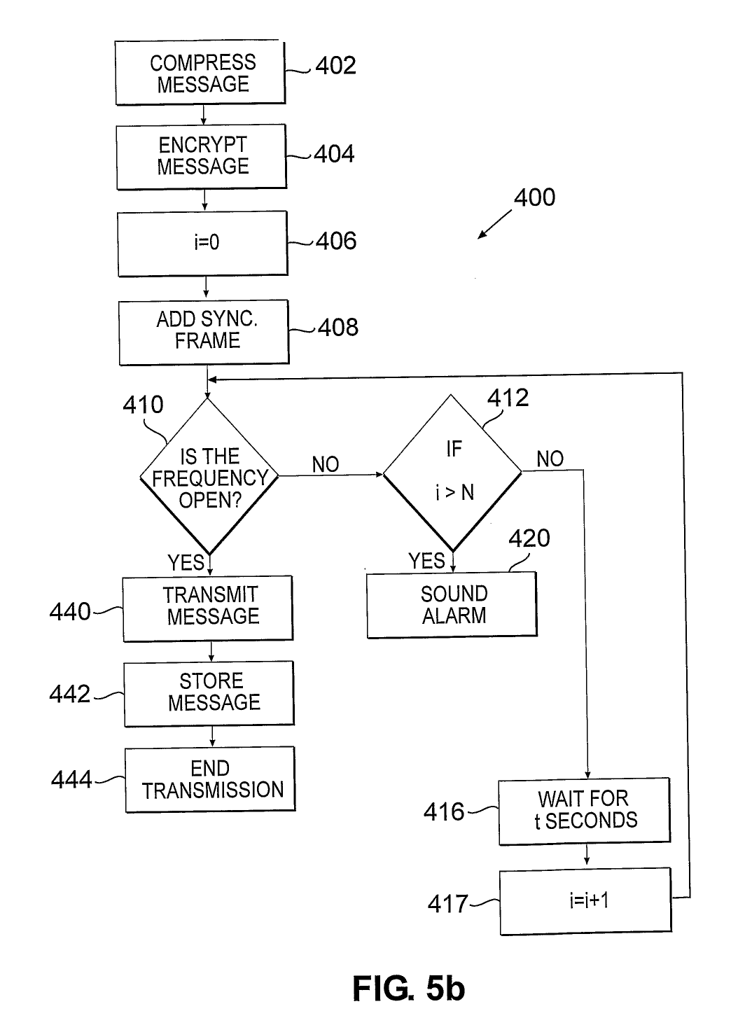

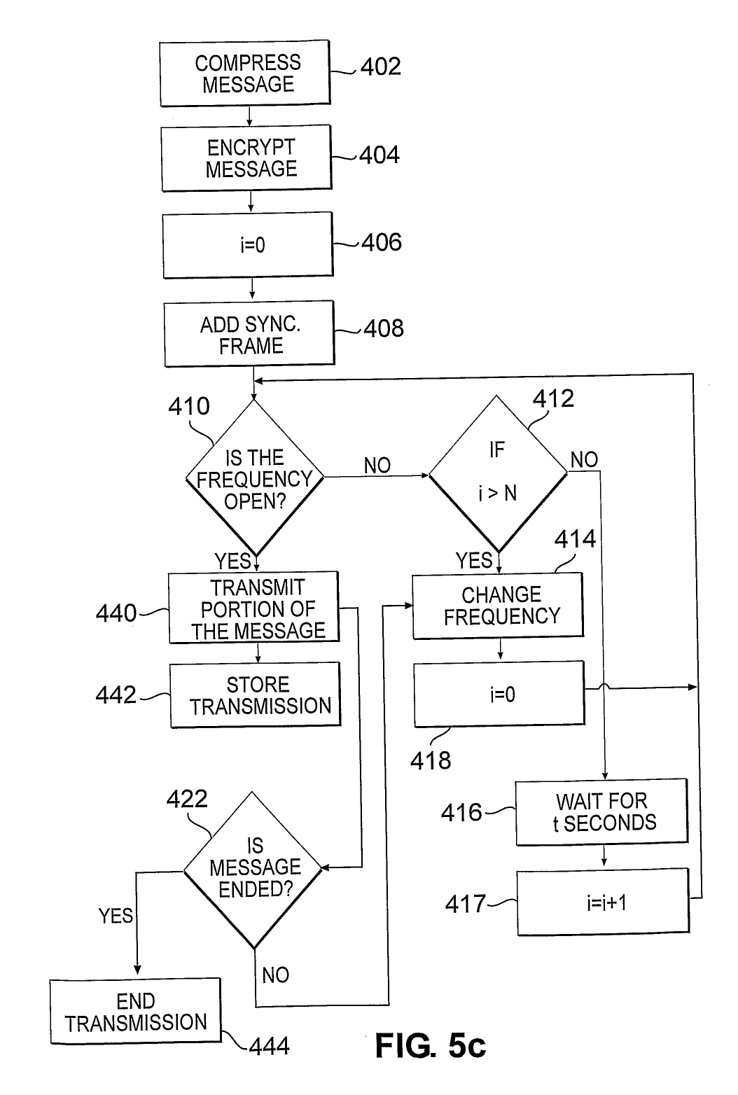

Referring to FIGS. 5a, 5b and 5c, flowcharts depicting an exemplary message processing procedure 400 performed on messages that are to be transmitted (e.g., the message 150, the message 51) are shown. Such messages are processed prior to being transmitted from one device, e.g., remote control unit 4000, dispensing unit 10 and/or sensor unit 30, to another device, such as remote control unit 4000, dispensing unit 10, and/or sensor unit 30. The procedure 400 may be implemented to enable wireless transmission using a reliable communication mechanism, such as “listen before talk”, as depicted in FIG. 5a, and/or frequency hopping, as depicted in FIG. 5c. Moreover, the procedure 400 may include a listen-before-talk feature, as depicted in FIG. 5b, and/or an alarm feature to indicate when a frequency is occupied (e.g., in use or jammed) for a given time.

As shown in FIGS. 5a-c, a message to be sent (e.g., between any of remote control unit 4000, dispensing unit 10, and sensor unit 30) is compressed 402 and encrypted 404. Compression and encryption may be performed in a manner similar to that described in relation to FIG. 4. Message compression and encryption may be implemented by first compressing and then encrypting the message or, alternatively, first encrypting and then compressing the message.

After preparing the message for transmission (e.g., by adding a synchronization frame 408), the transmitting unit (e.g., remote control unit 4000, dispensing unit 10, and/or sensor unit 30) determines 410 whether the desired frequency is open for transmission (i.e., whether a first frequency can be used to transmit a message). This determination can be performed by listening to the desired frequency before transmitting. If the frequency is jammed (e.g., used by another device), the transmitting unit device (remote control unit 4000, dispensing unit 10, and/or sensor unit 30) determines 412 how much time or how many attempts to transmit on that frequency have failed using. The number of transmission attempts can be tracked using, for example, a counter, such as the counter “i”, as depicted at 406.

In some embodiments, the listen-before-talk operations are performed by measuring (detecting) the radio frequency interference (RFI) associated with the first frequency. Such detection can be performed, for example, by measuring the level of radiation present at that frequency. A high level of such radiation would be indicative that this frequency already includes significant levels of electromagnetic activity and interference, and thus transmitting the message in that frequency may result in a relatively high likelihood of erroneous and/or corrupt transmissions. The measured level of RFI is compared to an associated RFI threshold. If, based on the comparison, it is determined that the detected RFI level is below the associated RFI threshold (indicating that the level of RFI is tolerable), the message, or parts thereof, may be transmitted using that first frequency. If, on the other hand, it is determined, based on the comparison, that the level of detected RFI exceeds the associated RFI threshold (thus indicating that there is relatively significant interference), a different frequency may be selected to transmit the message. The second frequency selected (e.g., if the detected RFI level exceeds the associated RFI threshold) may also be tested in a similar manner to the first frequency to determine if the message can be transmitted through that frequency (based on measured RFI levels in that second frequency). The threshold associated with the second frequency may be the same or different from the RFI threshold associated with the first frequency.

In some embodiments, the RFI threshold associated with one or more frequencies may be adjustable according to one or more of: time elapsed from processing a message and transmitting it, the message type, number of failed transmissions, number of corrupted messages transmitted over time through the associated one or more frequencies, time interval in which the detected RFI is above the associated RFI threshold level, transmission power, bandwidth, modulation technique and the bit rate of the transmission. There may be other factors that affect an adjustable RFI threshold associated with one or more frequencies.

If it has been determined that the desired frequency is not open and that the number of attempts to transmit the message on that frequency exceeds a predetermined value N (e.g., N is a natural number), the current frequency used to transmit the message is changed 414 (as depicted in FIGS. 5a and 5c) to select a different frequency based, for example, on some predetermined frequency selection pattern. As shown in FIG. 5b, in some embodiments, a warning sign may be produced 420 (e.g., an audio alarm, a visual alarm, an email is sent, etc.) to indicate that attempts to transmit on the current frequency have failed. In circumstances in which a different transmission frequency is selected, the counter i is reset to 0.

If, on the other hand, it is determined that the number of attempts is less than the predetermined number (e.g., i<N), the transmitting unit waits 414 for a pre-defined period of time, e.g., t seconds (where t can be any non-negative number) before attempting to retransmit the message. In some embodiments, the predetermine number of transmission attempts on a particular frequency, N, is set to 2, thus allowing three (3) trials for each frequency. In some embodiments, the pre-defined wait period, t, is set to 0.1×10⁻³ seconds, in which case the time interval between each transmission attempt is 0.1 milliseconds.

After waiting a period of t seconds following an unsuccessful attempt to transmit, the counter i is incremented (e.g., i is incremented by 1) to represent another failed attempt to transmit at a specific, desired frequency.

If it is determined that the particular frequency selected is open and available for transmission of the message, the message (e.g., message 150 or 51) is transmitted 440 from one device (e.g., remote control unit 4000, dispensing unit 10, and/or sensor unit 30 depicted, for example, in FIGS. 1a-c) to another device (e.g., the remote control unit 4000, the dispensing unit 10, and/or the sensor unit 30).

As will become apparent below, and with reference to FIG. 5c, in embodiments in which the message includes separately transmitted portions, a determination is made 422 of whether the entire message has been transmitted (e.g., if there is no more data ready for transmission). If it determined that additional data, or portions of data, remain to be communicated, the operations for transmitting the remaining portions, including operations to select a transmission frequency and determine if that frequency is open, are performed.

Transmitted message(s) are stored 442 by the transmitting device. In some embodiments, the receiving unit stores the messages. In some embodiments, the message is stored by both the transmitter and the receiver. The stored message may be stored using any type of storage mechanism and in any format, such as in a flat-file, using a relational database (e.g., SQL), using a hierarchical data model (e.g., Adabas provided by Software AG), or any other database format or model, to facilitate tracking a patient's medical condition, validating product quality, and establishing a personal profile of a user for better prediction of patient's needs. The personal profile represents a pattern of usage of the fluid delivery device such as the fluid delivery device depicted in FIGS. 2a and 2b. Moreover, that pattern may be established by analyzing the database of received messages (e.g., using statistical and numerical approaches to analyze the database).

Referring to FIG. 6a, a flow diagram of exemplary operations to implement lossy compression and frequency hopping is shown. In using frequency hopping, one or more messages (e.g., message 51) are divided into several sub-messages that are transmitted over different frequencies. By way of a representative example to illustrate the operations of the flow diagram of FIG. 6a, the message 51 may include the letters “A” to “T”. That message may then be compressed using a lossy compression technique. During the lossy compression the letters “I” and “N” are dropped (represented by empty cells in buffer table 53). The compressed data is buffered by placing a sequence of five letters in a row, thus creating a buffer 53 that includes four rows. The compressed message is read column-wise from buffer 53, thus creating four segments 561 containing four letters each and one segment 562 (i.e., corresponding to the column of the buffer 53 that contained the missing letters “I” and “N”) having only two letters.

The compressed segments 56 are provided to, for example, a transceiver 50 included within a device or its units (e.g., remote control unit 4000, dispensing unit 10, and/or sensor unit 30). The transceiver 50 may perform additional data compression, such as lossless or lossy data compression, potentially resulting in some additional loss of information (if lossy compression is performed). In some embodiments, the transceiver 50 may be implemented as a standard communication device, such as Chipcon 2500 (available from Chipcon).

The compressed segments 56 may be transmitted on one frequency or may be transmitted on different frequencies as shown in FIG. 6a. The messages are divided in such a way that sequential bits of information are transmitted in different transmissions.

In some embodiments, the frequencies applied may be around 2.4 GHz. For example, a transmission 501 may be at 2.48 GHz, a transmission 502 may be at 2.41 GHz, a transmission 503 may be at 2.48 GHz, and a transmission 504 may be at 2.4 GHz. Using several frequencies to transmit the same message (or portions of the same message) increases the reliability of the transmission of the message, such as message 51. For example, even if part of message 51 is lost or corrupted, message 51 may still be decoded and corrected. Lost or corrupted transmissions are common phenomena as there are many transmissions on the same frequencies. Thus, the probability of receiving an incomplete or corrupted message is high. For example, the transmission 501, transmitted at a frequency of 2.48 GHz, includes a preamble (that is transmitted first) followed by the segment 562, namely “S” and “D.” Similarly, the transmission 502, transmitted at 2.41 GHz, includes data corresponding to the first column of buffer 53 (e.g., “A”, “F’, “K”, and “P” of the segment 561).

Furthermore, frequency hopping provides a transmission of more compact messages since there is no need for a synchronize frame for every frequency. For example, one of the transmissions, e.g., 501, of message 51 may carry one or more data values and a preamble (denoted as “PE” in FIG. 6a), and the other transmissions 502-505 of message 51 may carry only data. The data may include op-code, time signature, dosage of the therapeutic fluid (in form of concentration, weight, active units, pump stroke and the like), level of body analyte (e.g. glucose level), information regarding the physical or medical condition of the patient (e.g., age, weight, blood pressure, heart rate, etc.), software or software updates (including firmware), control signals and the like. The transmissions may include frame synchronization, error detection code (such as a cyclic redundancy check (CRC)), telemetry identifier, clock synchronization, and other such information.

Referring to FIG. 6b, a flow diagram of exemplary processing operations performed on the received transmissions of a segmented message 51 is shown. The transceiver 50 at a receiving unit (e.g., remote control unit 4000, dispensing unit 10, and/or sensor unit 30) is configured to receive one or more transmissions at a given set of frequencies (e.g., a predetermined set of frequencies). The transmissions are combined by the receiving unit (e.g., remote control unit 4000, dispensing unit 10, and/or sensor unit 30) to form a data block 52. In circumstances in which lossy compression was performed on the original data segmented that was to be transmitted, the data block 52 may be smaller than the original message (e.g., block 51 at FIG. 6a). Data block 52 may also be smaller than the original block 51 due to transmission errors and corruptions that may have occurred. Although part of the message may have been lost, the message can still be processed (and decoded) by the receiver (e.g., remote control unit 4000, dispensing unit 10, and/or sensor unit 30). Buffering the message 52 may be one of the ways in which the effects of a lost transmission may be minimized as shown in FIG. 6b. The message may be written to a buffer in a specific pattern and read from the buffer in a different pattern (e.g., each significant portion of information is transmitted in different transmissions). This form of buffering may be referred to as an interleaver or a deinterleaver. The lost data may also be recovered using an error correction code.

The data block 52 is decompressed to form data line 54, which, in the example used, comprises the letters from “A” to “T”, but excluding the letters “B”, “G”, “L” and “Q”, which, in this example, were lost in transmission 505. In the some embodiments, lost data may be recovered through error correction processing, which may be performed on the original message 51.

In some implementations, an error correction code may be used on the message and processed at the receiving unit (e.g., remote control unit 4000, dispensing unit 10, and/or sensor unit 30) to detect and correct any errors in the message. The error correction code may be of any form, and in some implementations, may be implemented as a Viterbi coder and/or a Viterbi decoder, or other types of error correcting coding implementation (e.g., Reed-Solomon error correction). In some embodiments, more than one error correction code may be applied to the message being transmitted. In some embodiments, a spread spectrum communications approach may be used to transmit messages among the various units of the therapeutic fluids dispensing device, e.g., the remote control unit 4000, dispensing unit 10, and/or sensor unit 30. By applying a spread-spectrum procedure to a message associated with an associated bandwidth, the resultant bandwidth of the processed message is expanded such that it is larger in size than the bandwidth of the original message.

The message's associated bandwidth is expanded so that the processed message occupies a resultant bandwidth that is larger in size (i.e., broader) than the original bandwidth of the message. Use of the spread spectrum technique thus decreases the potential interference and corruption of the message, while increasing the achieved communication security and privacy for transmitting the message. The spread spectrum and frequency hopping are methods of transmitting radio signals by rapidly switching a carrier among many frequency channels, using a pseudorandom sequence known to both transmitter and receiver. The spread spectrum signal stature technique may include applying direct sequence to locally generated pseudo noise code to encode the data of the message to be transmitted, frequency hopping, or a hybrid of direct sequence techniques or frequency hopping techniques (which can be used for multiple access and/or multiple functions).

Typically, the initiation of frequency hoping communication is as follows:
1. The initiating party sends a request via a predefined frequency.
2. The receiving party sends a number, known as a seed.
3. The initiating party uses the seed as a variable to determine the sequence of frequencies that is to be used. In some embodiments, the period of the frequency change is predefined.
4. The initiating party sends a synchronization signal via the first frequency in the determined sequence, thus indicating to the receiving party that it has determined the sequence.
5. The communication begins, and both the receiving and the sending party change their frequencies based on the determined order, commencing at the same point in time.

In some embodiments, a predefined frequency-hopping sequence is determined separately (not related to whether there is a message to be transmitted), and/or predetermined. In some embodiments, the sequence of frequencies that is to be used is determined based on the seed. In some embodiments, spread spectrum uses a sequential noise-like signal structure to spread the normally narrowband information signal over a relatively wide band radio frequencies. Under these circumstances, the receiver correlates the received signals to retrieve the original information signal.

In some embodiments, spread spectrum telecommunications can be applied by frequency-hopping spread spectrum (FHSS), direct-sequence spread spectrum (DSSS), time-hopping spread spectrum (THSS), chirp spread spectrum, or CSS, in which a frequency varying signal (e.g., a sinusoidal signal whose frequency changes as a function of time) is modulated to encode the message, and combinations of one or more of FHSS, DSSS, THSS, and CSS. As described herein, orthogonal frequency division multiplexing may also be included as a form of spread-spectrum.

In some embodiments, spread spectrum telecommunications may be implemented as Ultra-wideband (UWB), as described, for example, in Wireless Ethernet standard IEEE 802.11.

In some embodiments, Code Division Multiple Access (CDMA) may be applied for transmission of messages (e.g., message 51 or 150). CDMA is a form of multiplexing and a procedure of multiple access that divides up a radio channel not by time (as in time division multiple access), nor by frequency (as in frequency-division multiple access), but by using different pseudo-random code sequences for each user. CDMA is a form of spread spectrum signaling since the modulated coded signal has a higher bandwidth than the data being communicated. CDMA can be applied according to IS-95 standard.

In some embodiments, an error correction code will be sent as part of the message. In some implementations, only the most important parts of the message are coded using error correction codes. Moreover, relatively important parts of a message may be sent repeatedly. For example, the dosage of a therapeutic fluid may be sent multiple times in one or more messages.

In some embodiments, message encryption, message compression, message storage, and logical message validation may be included in any of the communication features of the remote control, sensor, or infusion pump.

In some embodiments in which error correction codes are used, message encryption, message compression, message storage, and logical message validation may be included in any of the communication features of the device (e.g., remote control unit 4000, dispensing unit 10, and/or sensor unit 30) that can dispense insulin according to monitored glucose levels, thus providing a closed loop system.

In some embodiments, the compression techniques used may implemented using a so-called “lossless” data compression technique or a so-called “lossy” data compression technique. In some embodiments, a simpler static code, such as unary coding, Elias gamma coding, MPEG protocol, Fibonacci coding, Golomb coding and/or Rice coding may be applied to provide some form of compression.

In some embodiments, the encryption process may be executed while the data is still in the memory matrix. The encryption can be implemented as a simple shuffle (e.g., reading the data from different starting points, such as reading by starting at the last row of the first column of the buffer). Furthermore, the reading of the compressed data may differ from the writing direction.

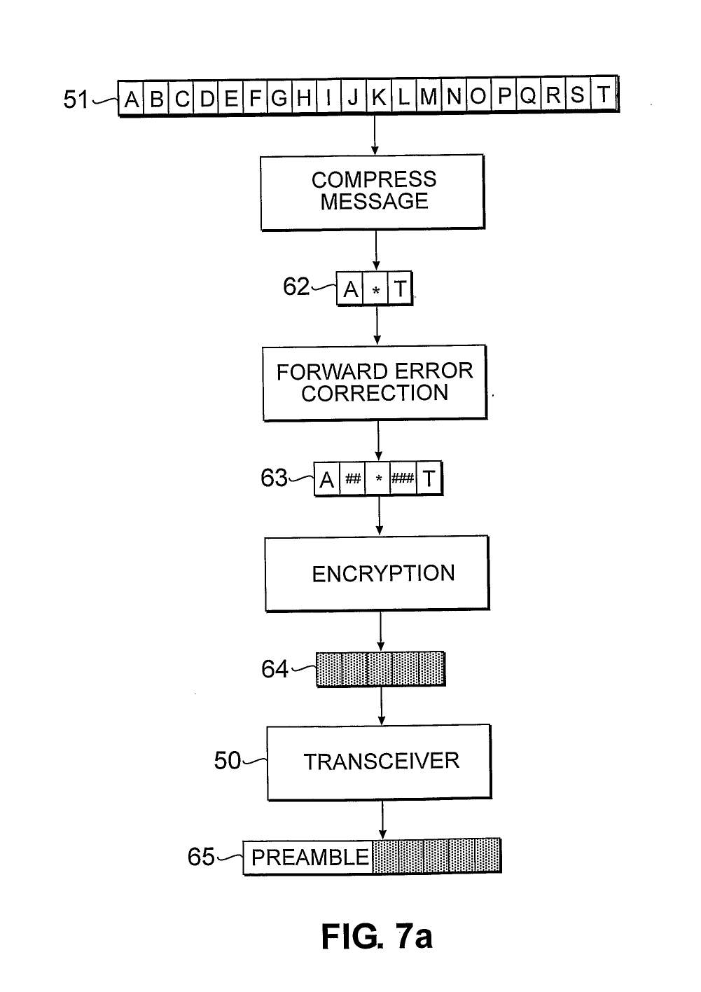

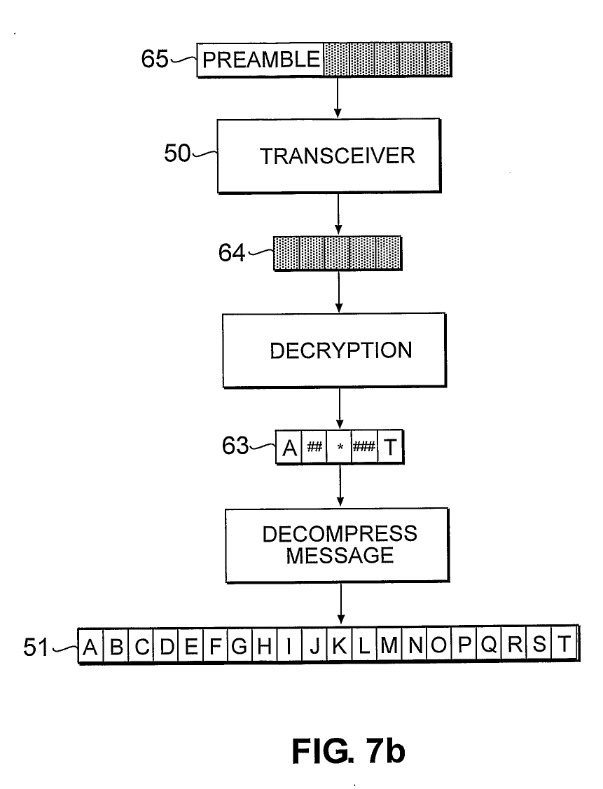

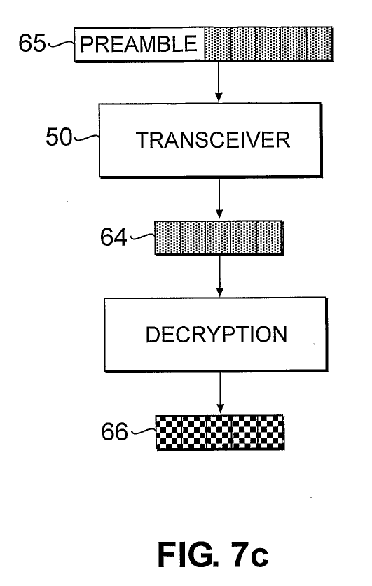

In some embodiments, the encryption may be performed before compression. Referring to FIGS. 7a, 7b and 7c, exemplary processing operations (e.g., compression, error correction and/or encryption) of a message 51 are shown. FIG. 7a depicts the processing stage of message 51 prior to transmission. As shown, the message 51, which includes the letters from “A” to “T,” is compressed, in some embodiments, using a lookup table (also referred to as a codebook or codebook compression) to “A*T”. The compressed data 62 is processed, in some embodiments, by a forward error correction procedure. The forward error correction codes are represented by the data two symbols “##” and “###” which are determined based on their respective preceding and succeeding characters (e.g., “##” is determined based on the characters “A” and “*”), thus resulting in data line 63. The compressed message 63 is encrypted. In some implementations, the encryption uses an encryption key that is related to the hash number assigned to the transmitting and/or receiving units. The encrypted message 64 is then transmitted by a transceiver 50. The transceiver 50, which may be located at remote control unit 4000, dispensing unit 10, and/or sensor unit 30, adds to the transmission a preamble and sends the transmission 65. The transmission 65 is then received by another device, such as a transceiver at remote control unit 4000, dispensing unit 10, and/or sensor unit 30.

Referring to FIG. 7b, a flow diagram of exemplary processing operations of the received encrypted transmission 65 at a device, such as a transceiver at the remote control unit 4000, dispensing unit 10, sensor unit 30, or any other portion of the fluid delivery device or an associated accessory (e.g., a wireless computer, a hand held computer, a smart phone, a personal digital assistance, and the like) is shown. In some implementations, the sending device (e.g., remote control unit 4000, dispensing unit 10, and/or sensor unit 30) and the receiving unit (e.g., remote control unit 4000, dispensing unit 10, and/or sensor unit 30) are paired, thus enabling each of the paired devices to work together and recognize each other's encryption keys. When transmission 65 is received by the receiving unit, the preamble of the transmission is removed by the transceiver 50, and the encrypted data 64 is decrypted, for example, using a decryption key that is related to the hash number assigned to the transmitting or receiving units. If a proper decryption code is applied during decryption, a proper compressed data 63 results. The compressed data message 63 may then be decompressed to reconstruct the original message 51.

Referring to FIG. 7c, a flow diagram of exemplary processing operations performed on the encrypted transmission 65 by the receiving unit (e.g., a device, such as remote control unit 4000, dispensing unit 10, and/or sensor unit 30) that received the message 65 is shown. When transmission 65 is received by the receiving unit, the preamble of the transmission is removed by the transceiver 50 at the device and the encrypted data 64 is decrypted. If an incorrect decryption code is applied during this stage, unusable data may be generated.

In some embodiments, an encryption key may be used for encrypting messages and decrypting messages between any pair of units (e.g., any two of the following: remote control unit 4000, dispensing unit 10, sensor unit 30, and the like). In some embodiments, the receiving unit and/or the transmitting unit may each have at least one decryption key and at least one encryption key. In some embodiments, the decryption key and encryption key may be identical. In some embodiments, the decryption key may include a fixed value, such as the serial number or the hash number of the dispensing device or of one of its units, it may include a value that is achieved during the pairing process, such as the fixed value (e.g., serial number or the hash number) of the other unit, it may include at least one variable value which may be a time dependent value (e.g., the date, hour, or the like), or may be changed by a defined pattern by one of the units using a predetermined pattern for the unit which will receive the message. In some embodiments, the variable value may be related to the message context, the message data, or to the number of transmissions. For example, in some embodiments, the variable value is the number of received messages. The variable value may be the average daily dosage or the number of the executed commands.

In some embodiments, the decryption key (or code) and/or the encryption key (or code) includes at least one variable value that is shared by more than one pair of units. In some embodiments, the decryption key includes a fixed value, such as the device serial number, a value that is achieved during a pairing process between units (e.g., the fixed value of the other unit, and at least one variable value, such as the number of verified transmissions). In some embodiments, there may be more than one decryption key, for example, one key for every group of messages. For example, the transmissions relating to the dosage of the therapeutic fluid will have the same decryption key, while the maintenance transmissions will have another key.

In some embodiments, the decryption key and/or the encryption key includes a fixed value, such as the unit serial number, a value that is achieved during pair process such as the fixed value of the other unit, and at least one variable value, such as the number of verified transmissions. These values can be chained together (concatenated). For example, the fixed number is 1234, the pairing number is 78, and the variable is 99. Thus, the so-called “chained” values can be treated as a sequence of digits of the same number, namely 12,347,899. These values (e.g. fixed and/or variable value) can also be multiplied by each other, divided, or processed in other ways to form an encryption key.

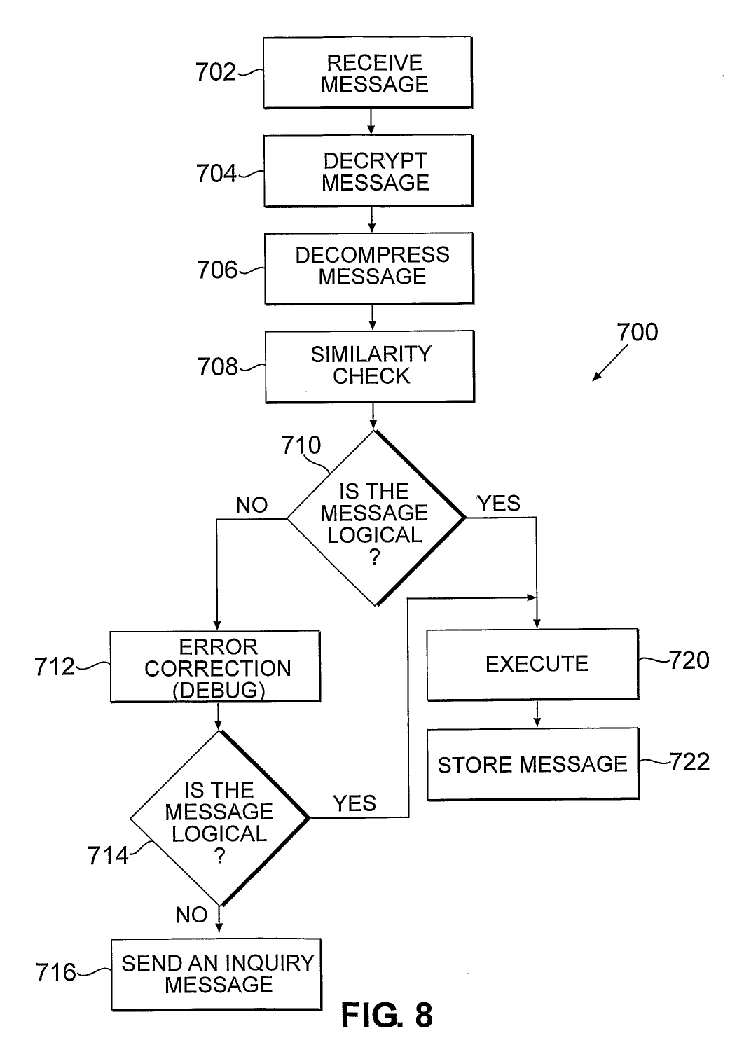

Referring to FIG. 8, a flowchart of an exemplary procedure to process an incoming message by a receiver located in the receiving unit is shown. The receiving unit receives 702 a message. The receiving unit then decrypts 704 and decompress 706 the received message. Data may then be extracted from the message.

A similarity check is then performed 708. The similarity check determines whether a command or data included in the message is consistent with stored or predetermined command or data. The degree of the data consistency can be set at a value less than 100%, thus enabling partially matching commands to be corrected and executed. The consistency of the data can be evaluated by simple comparisons, fuzzy logic, dynamic logic, predictive analytics, time series models (e.g., autoregressive models (AR) and moving average (MA) models), and other statistical processes.

If it is determined 710 that data of the message is logical (i.e., it matches a stored or predetermined command or data value), the message is executed 720 and stored 722.

If it is determined 710 that the message is not logical (i.e., it does not match a stored or predetermined command or data value), the command or data (included in the received message) undergoes some error correction at 712 to, for example, recover at least some missing data and/or errors in the received message to provide at least some of original content of the message. If the message is corrected and is determined to be logical (i.e., it matches a stored or predetermined command or data), the command or data (included in the message) is then processed (executed 720 and stored 722). Otherwise, an inquiry message is sent 716 to the device transmitting the message.

Thus, for example, the extracted data may be checked by a logical function, process or component. This procedure may be based on previous knowledge included or gathered by the unit. For example, the receiver at the device may have a database of possible commands, and each command may be significantly different for the other commands by name, length, parameters, the names of the parameters, and so forth. If the data received does not correspond (e.g., match or resemble) to any command in the database, a message is sent to the transmitting unit to resend the message because the received data does not appear to correspond to any valid command listed in the database.

If the message includes data (e.g., a command or a data value) matching an existing command or data value, then the command or data value may be processes (e.g., executed). If there is not a perfect match but some similarity to a known command stored in the database of valid commands, the receiver of the message may attempt to correct the message. The error correction may be done by extracting data from the message or from the pattern of usage of the device. For example, a non-logical message can be a message that is irregular in terms of time or parameters for the user. The irregularity is determined based on data stored during usage, e.g., unusually specified high or low dosages, a high dosage instead of a regular low dosage previously used for the patient (for example, a specified high dosage of 15 units of insulin where typically a low dosage of 1-5 units of insulin may have been used for the patient, may be deemed to constitute not logical data).

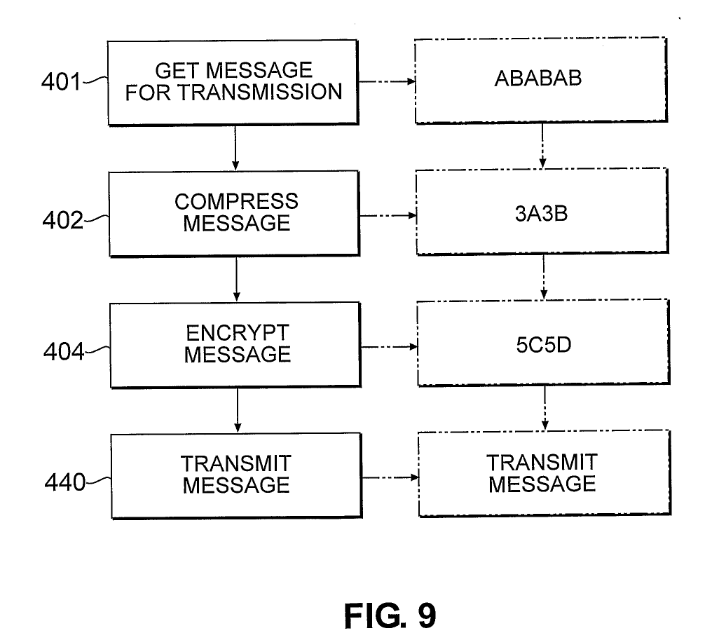

Referring to FIG. 9, a flow diagram of exemplary processing operations of a message transmitted by a unit (e.g., remote control unit 4000, dispensing unit 10 or sensor unit 30) is shown. The left column (401, 402, 404, and 440) includes the processing operations applied to the message to be transmitted by the unit and the right column shows the resultant intermediate messages as at corresponding points in the sequence of the operations 401, 402, 404, and 440.

As shown, the original message is retrieved 401 by the unit. In this example, the original message is “ABABAB.” The message is compressed 402, resulting in the intermediate data message represented as “3A3B.” The compressed message is then encrypted 404. In the example of FIG. 9, the ASCII code value of each text character is increased by two, thus resulting in the intermediate data message represented as “5C5D.”Additional processing operations may be performed on any of the original and/or intermediate message before the actual message to be transmitted is transmitted 440.

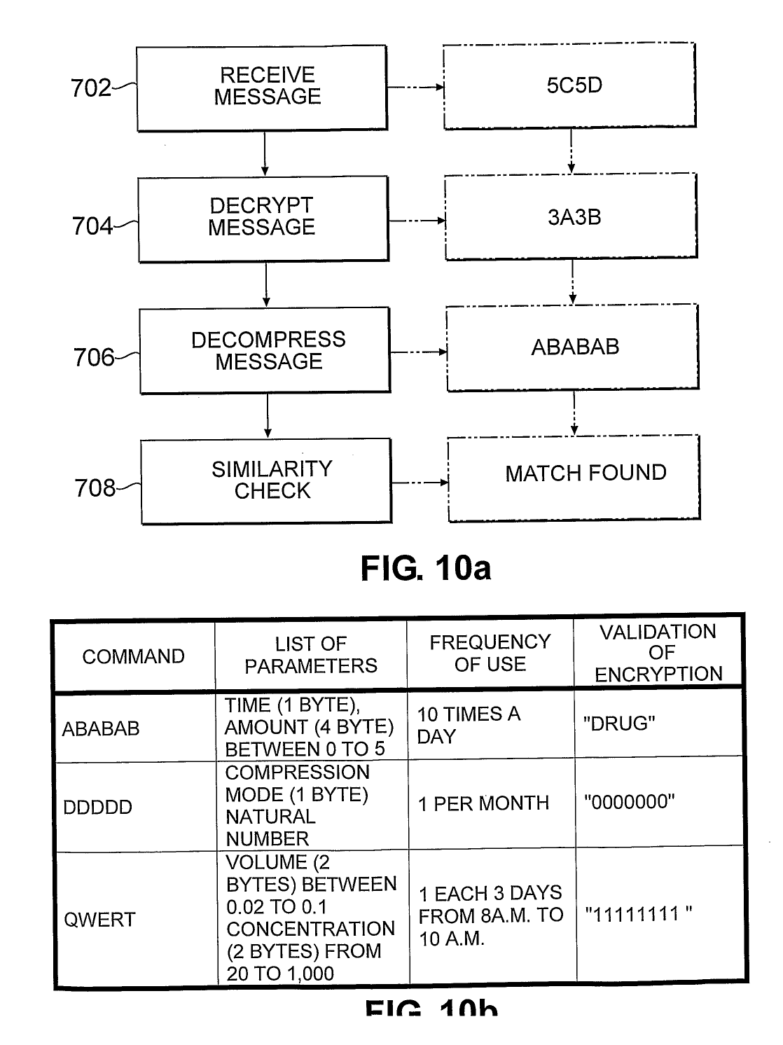

Referring to FIG. 10a, a flow diagram of exemplary processing operations for receiving a message at a receiving unit is shown. The left column includes operations 702, 704, 706 and 708 to process a message received at the unit. The right column represents resultant intermediate messages at various points corresponding to the processing operations. Thus, as shown, the message, represented as “5C5D”, is received 702. The received message is decrypted 704, resulting in the intermediate message represented as “3A3B,” and this resultant intermediate message is then decompressed 706, resulting in the message represented as “ABABAB.” The decompressed message (or data/commands included within the message) is used to determine 708, through a similarity check (which may be a check like that described in relation to FIG. 8), if the decompressed message, or its content, matches valid commands and/or data stored in a database located on a storage device of the receiving unit. If a match is found, and the message includes command(s), the command(s) may be executed.

Referring to FIG. 10b, a schematic diagram of an exemplary database that may include information regarding valid command is shown. The database comprises three (3) commands, with each command having, in this example, three (3) characteristics that are detailed in the corresponding row of the table. As further shown, the table has four columns: the command name (labeled “command”); a list of parameters and their values associated with the respective commands (that column is labeled “list of parameters”); descriptions of how often the command(s) are used or received (labeled “Frequency of use”); and a validation of encryption code (labeled “validation of encryption”) that may be used to validate that the encryption was performed correctly (e.g., the code to be validated may be an integral part of the command, added to the command, may have a standalone meaning, and may include a recognizable pattern).

In some implementations, one or more databases may be stored on each device. For example, each of the remote control unit 4000, dispensing unit 10, reusable part 100, and other such modules of the system, may include a storage mechanism, such as a database, for storing messages (e.g., data and/or commands) transmitted to another device, for storing incoming messages, and for storing a logbook for incoming and outgoing messages. As the string “ABABAB” is considered a valid command (or a valid data value in the received message), that command may be executed or the data can be further analyzed to correct any errors that may occur during the transmission. As further shown in FIG. 10b, the command “DDDDD” has one parameter, which can be a natural number and which may be used to facilitate the receiver to decode the transmission as it expect to receive a defined format of transmission for this command. The frequency of use is primarily intended for logical examination of the data received. For example, if the command “DDDDD” was received twice a week, it might be considered an illogical transmission if received ten (10) times in a single day. In another example illustrating a determination/identification of an illogical message, if the command “ABABAB” was received only five (5) times in a day, this may be indicative that there may have been a problem in the transmission of the command. As a further example, the 11111111 validation code of the command “QWERT” is a bit pattern which is expected to be at a specific location of the encrypted transmission (e.g., application of a different combination of encryption and compression processes than that expected will result in a different bit pattern being generated, or in the specified bit pattern being located in a different location than that expected in of the resultant data stream).

Referring to FIG. 10c, a flow diagram of exemplary processing operations performed on a message at a receiving unit (such as remote control unit 4000, dispensing unit 10, sensor unit 30, and the like) is shown. A message is initially received and processed in a manner similar to that described, for example in FIGS. 9 and 10a.

For example, the message may include a data value, such as a command “DDDDD”. The command “DDDDD” may typically be associated, as indicated by a database such as the database depicted in FIG. 10b, with a parameter that may be, in some embodiments, a natural number. However, as shown in FIG. 10c, processing of the receive message yields a processed message represented as “DDDDD . . . V” (in other words, the parameter associated with the processed message is a letter rather than a number). Implementation of error correction operation may enable recovery (for example, at 712) of the correct parameter based on the resultant parameter derived from the received and processed message. Specifically, in some embodiments, an assumption may be made that one possible reason for obtaining an invalid parameter is a bit transition error that occurred during transmission. For example, the binary code for “V” is 01110110 and the binary of the digit 6 code is 00110110. Thus, the receiving unit may correct the invalid message parameter byte corresponding to the command “DDDDD” to a valid parameter. In some embodiments, when a mismatch between the expected data to the received message occurs, as determined, for example, at 710, an analysis of the stored data regarding the profile of the usage may be performed (also at 710) by applying a prediction by partial matching (PPM) technique to the recovered data of the received/processed message, or by any other mechanism to perform data validations. In this example, the received/processed message did not match the formatting profile corresponding to the recovered command. Thus, in some embodiments, an inquiry message may be sent to request retransmission of the message.

Also shown in FIG. 10c is the similarity check 708 that determines (e.g., by performing a comparison) whether the received/processed data string “DDD” (resulting after the decompression operations performed at 706) corresponds to a string stored, for example, in a database. Because the resultant message is similar to the command is “DDDDD” stored on the database of the receiving unit, under these circumstances, a correction from “DDD” to “DDDDD” may be performed (at 712).

In some embodiments, a validation of the decryption and decompression processes may also be performed, and in some cases, such validation is performed before error correction is performed. If a wrong encryption key or encryption techniques were applied, or a wrong procedure for decompression was used, the result may be (in some situations) that a message from other equipment would be considered a legitimate command, or would otherwise be corrected (by the error correction module) to recover a valid command. Therefore, it may be useful, when a compression and an encryption operation are performed, to also perform a validation process with respect to the decompression and decryption operations.

In some embodiments, the validation process may include comparing a predefined part of the message with a known bit combination that may be a symbol, a string, or a number. The validation process may be general to the commands associated with all the units or it may be unique. For example, the commands shown in FIG. 10b may be used by all units as detailed in the table. Therefore, validation of any of the commands may generally be performed by all the units. If the validation code also includes the serial number of the transmitting unit, the serial number may be used to perform unique validation because every unit has a unique serial number. For example, the command “DDDDD” (FIG. 10b) may require a validation string of “0000000” in the predetermined part of the message before a determination that the received “DDDDD” command is valid, and any other result for this validation operation may indicate a problem in the decryption or decompression process that resulted in the command “DDDDD”.

In some embodiments, one or more error correction modules may be included in each of the devices (e.g., remote control unit 4000, dispensing unit 10, reusable part 100, sensor unit 30, and the like).

In some embodiments, the data validation can be performed by another device, such as a personal computer, hand held computer, a cellular phone, or other medical device. In some embodiments, the original message (i.e. before processing), or any part thereof, may be stored. In some embodiments, a decision trace back, such as a Viterbi decoder comprising a FILO (first-in-last-out) buffer, may be used to reconstruct data in a correct order. In some embodiments, the original message may be parallel processed for error corrections, although a sequential, serial approach may be used as well. In any case, processing of the message for transmission or reception as described above may be performed on different parts of the message (e.g., header, data, command, etc.) or the whole message. In some embodiments, the original message can be processed by a sequence of procedures (implemented by software and/or hardware) for error corrections. Processing of the message for transmission or reception as described above may be performed on different parts of the message (e.g., header, data, command, etc.) or on the whole message.

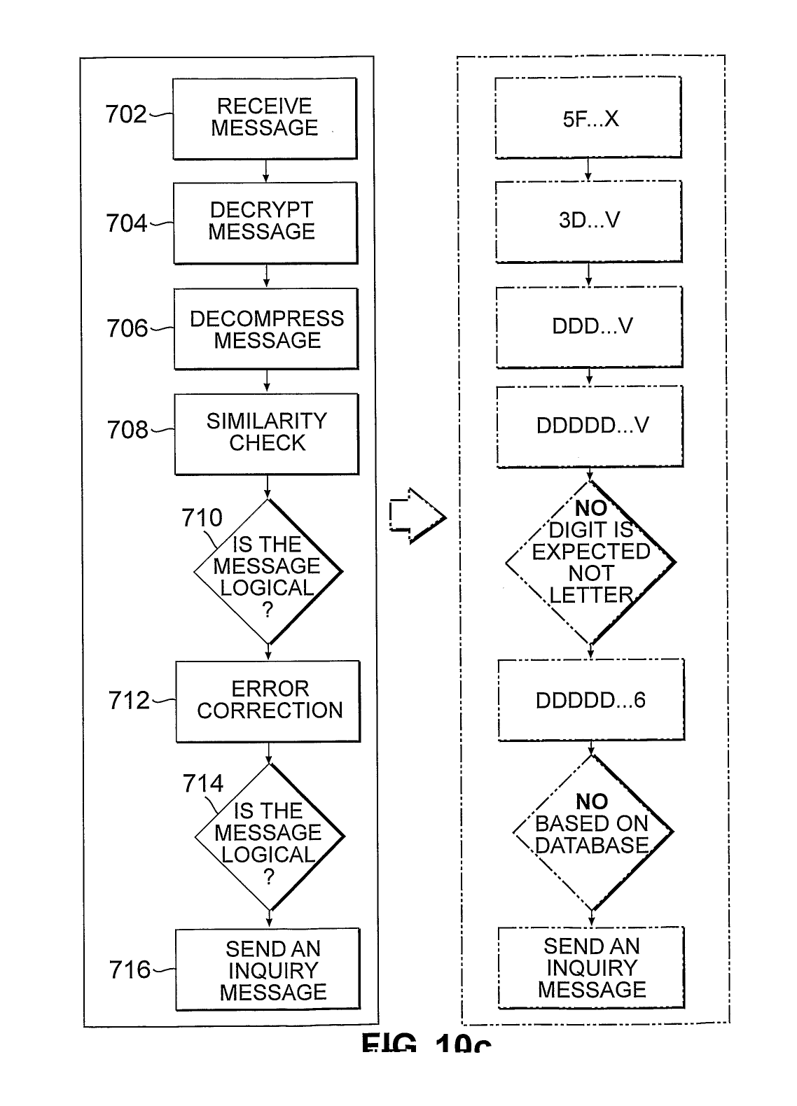

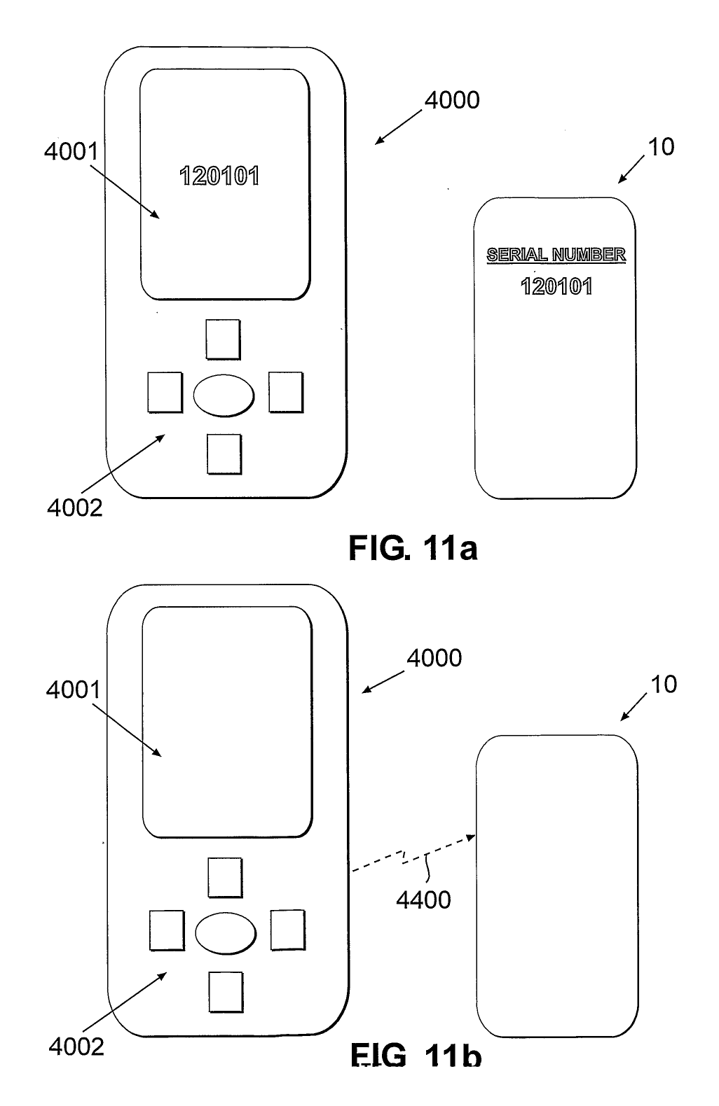

Referring to FIGS. 11a and 11b, schematic diagrams of exemplary devices implementing the so-called “pairing” process and the initiation of an encryption key/decryption key processed by the paired devices are shown. FIG. 11a depicts the first stage of the pairing process. The serial number of the dispensing unit 10 is typed, or otherwise manually entered, into the remote control unit 4000 by a keypad 4002, as shown on the remote control display 4001. It may be advantageous that the serial number or the encryption key remain private (and thus not transmitted) to prevent interception or corruption of the transmission including the key or serial number.

The remote control unit 4000 unit may use the serial number of the dispensing unit 10 to determine the encryption key it would use for transmitting messages and the decryption key for received messages. In some embodiments, there will be other parameters, such as time stamps, the device internal serial number, and number of transmissions that may be used to determine the encryption key and the decryption key. Thereafter, the paired devices can be used to perform secure communications by encrypting and decrypting messages transmitted between them.

In some embodiments, the remote control transmits, using a transmission 4400, the encryption key and/or the decryption key (or parts of them) to the dispensing unit, as depicted in FIG. 11b.

In some embodiments, the dispensing unit can transmit or receive open broadcast. The open broadcast may be an unencrypted transmission or an encrypted with generic encryption key.

In some embodiments, the encryption key of the remote control may be different from the decryption key of the dispensing unit. In some embodiments, the encryption key of the dispensing unit may be derived from the decryption key of the remote control. In some embodiments, the decryption key of the dispensing unit is derived from the encryption key of the remote control. The encryption key or decryption key of the remote control or parts of them may be manually typed or inputted by the patient.

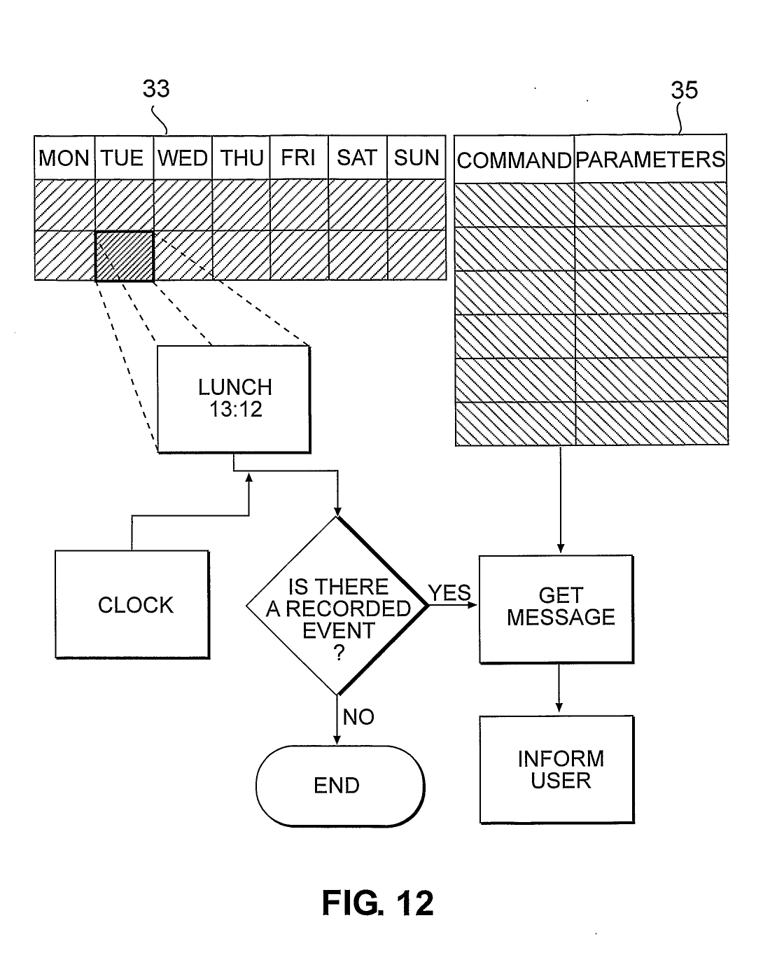

Referring to FIG. 12, a diagram depicting an exemplary logical prediction feature is shown. The logical prediction process is applied to a message to validate the message so as to determine if the message or action was not initiated in the usual time or circumstances. For example, if data for a particular patient indicates that the patient usually had lunch on Tuesday 13:12, the device can remind the patient this event in a predetermined time interval, such as 10 minutes before the event (at 13:02), or it can notify the patient if the corresponding commands had not been provided, in this case, a bolus should have been dispensed before lunch.

To support the logical prediction feature, the data stored at a device (e.g., in the database 33 which may be included at any unit of the dispensing device) may be compared to the time, as indicated by data provided by a clock associated with the device remote control unit 4000, the dispensing unit 10 and/or the sensing unit 30). In some embodiments, the device may include an internal clock (e.g., a counter, timer, and the like). Alternatively, the time may be obtained from an external clock. The database 33 may also include so-called “raw data” (e.g. not processed for transmission) but enhanced results may be obtained when the data is processed by numerical techniques, such as a predictive processes (e.g., learning processes). Examples of such numerical techniques include the ID3 technique, Gaussian Linear Discriminant, Gaussian Quadratic, Gaussian Mixture Classifier, K-Nearest Neighbor, Binary Decision Tree, Parzen Window, Histogram, Naive Bayes, Support Vector Machine, etc. In some embodiments, the results of the learning processing are provided to a program code, or, alternatively, the program code may be provided to database 33. The database can be stored in a hard drive, a non-volatile random access memory, a random access memory (RAM), or in any other storage mechanism.

If an action is supposed to be taken at a specific time frame, the relevant message details may be loaded from the archive of stored messages 35. The user is notified about the action or the message, and the device waits for the user approval. For example, actions are supposed to take place in the infusion pump (e.g., dispensing unit 10) or in the sensor. When that is the case, messages advising the patient about the action that will be taken upon the patient's approval may be sent to the remote control for user approval. In some embodiments, entire communication sequences that are meant to be performed between the parts of the device or between different devices are stored in one database.

In some embodiments, any of the devices, such as the remote control unit 4000, the dispensing unit 10, and/or the sensor 30, may include one or more of the following: an error correction module to perform error correction, a message encryption module to code messages to be transmitted, a message compression module to compress messages that are to be transmitted, and a logical message validation to, among other things, determine whether valid messages have been received/processed.

The systems and methods disclosed herein may be implemented using, for example, a data processor, such as a computer that also includes storage devices that store, among other things, a database system, digital electronic circuitry, firmware, software, or any combinations of them. Moreover, the above-noted features and other aspects and principles of the present disclosed embodiments may be implemented in various environments. Such environments and related applications may be specially constructed for performing the various processes and operations according to the disclosed embodiments or they may include a general-purpose computer or computing platform selectively activated or reconfigured by code to provide the necessary functionality. The processes disclosed herein are not inherently related to any particular computer, network, architecture, environment, or other apparatus, and may be implemented by suitable combinations of hardware, software, and/or firmware. For example, various general-purpose machines may be used with programs written in accordance with the disclosed embodiments, or may be implemented using specialized apparatus or system to perform the required methods and techniques.

The systems and methods disclosed herein may be implemented as a computer program product, i.e., a computer program tangibly embodied in an information carrier, e.g., in a machine readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Although some of the processes described herein are depicted in a particular order, the processes may occur in other orders as well. Moreover, in some implementations, one or more aspects of the processes may be omitted.

The foregoing description is intended to illustrate but not to limit the scope of the invention, which is defined by the scope of the appended claims. Other embodiments are within the scope of the following claims.

1.-278. (canceled)

279. A method for communicating between at least one unit of a therapeutic fluid dispensing system and at least one other unit of the dispensing system, the method comprising:
providing the at least one unit and the at least other unit, wherein the units are selected from the group consisting of: a pump, a remote control for controlling at least the operation of the pump, and an analyte sensor;
determining whether a first frequency is available for transmission of a message related to operations of the dispensing system, wherein said determining includes performing a comparison of detected radio frequency interference (RFI) associated with the first frequency to an associated RFI threshold; and
transmitting the message based on the determination.

280. The method of claim 279, wherein the RFI threshold is adjustable according to one or more of:
time elapsed from processing the message and transmitting it,
message type,
number of failed transmissions,
number of corrupted messages,
time interval in which the detected RFI is above the associated RFI threshold level,
transmission power,
bandwidth,
modulation technique, and
the bit rate of the transmission.

281. The method of claim 279, wherein transmitting comprises transmitting the message using the determined first frequency if the detected RFI is below the associated RFI threshold and a second frequency if the detected RFI is above the associated RFI threshold.

282. The method of claim 279, further comprising:
determining a second frequency to transmit the message in response to the determination that first frequency cannot be used to transmit the message.

283. The method of claim 279, further comprising:
generating one or more user-notifications in response to the determination that the first frequency cannot be used to transmit the message.

284. The method of claim 279, further comprising:
encrypting at least one portion of the message.

285. The method of claim 284, wherein encrypting the at least one portion of the message comprises:
using at least one transmitting encryption key, the transmitting encryption key enabling the message to be recognized by one or more of the units.

286. The method of claim 285, wherein the transmitting encryption key comprises a variable encryption key or a fixed encryption key.

287. The method of claim 285, further comprising:
receiving the transmitted message; and
decrypting the received message using at least one receiving encryption key.

288. The method of claim 287, wherein the at least one receiving encryption key includes one of the at least one transmitting encryption key.

289. The method of claim 287, wherein the at least one receiving encryption key includes a receiving encryption key different from the at least one transmitting encryption key.

290. The method of claim 279, further comprising:
compressing at least part of the message to reduce message length.

291. The method of claim 290, further comprising:
encrypting the at least part of the message.

292. The method of claim 279, wherein transmitting the message comprises:
transmitting the message wirelessly using a spread spectrum protocol from the at least one unit of the dispensing system to the other unit of the dispensing system.

293. The method of claim 279, further comprising:
interleaving the message before transmitting the message.

294. The method of claim 279, further comprising:
coding the message to enable error correction of the message.

295. The method of claim 294, wherein coding the message to enable error correction of the message comprises:
coding the message using Viterbi-related error-correction coding schemes.

296. The method of claim 279, further comprising:
receiving the transmitted message.

297. The method of claim 296, further comprising:
performing a similarity check to determine whether the received message is valid based, at least in part, on stored information representative or message content.

298. The method of claim 296, further comprising:
decoding the message such that at least some missing data and/or errors in the received message are recovered to provide at least some of original content of the transmitted message

299. The method of claim 279, wherein the message related to operations of the dispensing system comprises one or more of: an op-code; a time signature; a dosage of therapeutic fluid representative of at least one of a concentration of a therapeutic fluid or analyte, a weight of the therapeutic fluid, an active units of the therapeutic fluid, a pump stroke of the therapeutic fluid; an amount of analyte; a glucose level; information regarding a condition of the patient; patient information representative of at least one of an age, a weight, a blood pressure, a heart rate; program code; control information or signals; a frame synchronization mechanism; an error detection code; a telemetry identifier; and a clock synchronization.

300. The method of claim 279, wherein transmitting the message comprises:
transmitting at least one portion of the message using a first selected transmission frequency and at least another portion of the message using a second selected transmission frequency.

301. The method of claim 279, wherein transmitting the message comprises:
storing, according to a first ordering pattern, portions of the message into a buffer;
retrieving, according to a second ordering pattern, the stored portions of the message; and
transmitting the portions of the message retrieved according to the second pattern in the order in which the portions were retrieved.

302. The method of claim 301 wherein storing, according to a first ordering pattern, portions of the message, comprises:
storing the portions of the message in the buffer according to a row-by-row pattern;
and wherein retrieving, according to the second ordering patterns, the stored portions of the message, comprises:
retrieving the portions of the message from the buffer according to a column-by-column pattern.

303. The method of claim 279, wherein transmitting the message comprises:
transmitting the message using a wireless communications protocol based on at least one of: Wireless Application Protocol (WAP), WiFi and WiMax.

304. The method of claim 279, wherein transmitting the message comprises:
transmitting the message using a wireless communications protocol based on Low Power Bluetooth protocol.

305. The method of claim 279, wherein transmitting the message comprises:
transmitting the message in the Industrial, Scientific and Medical (ISM) radio bands.

306. The method of claim 305, wherein transmitting the message in the ISM radio bands comprises:
transmitting the message in a radio band corresponding to frequency of between 2.4 to 2.5 Giga Hertz.

307. The method of claim 279, wherein the at least one unit is one of: an insulin pump and a glucose sensor, and the at least another unit is a remote control.

308. A method for communicating between at least one unit of a therapeutic fluid dispensing system and at least one other unit of the dispensing system, the units comprising an insulin pump, an analyte sensor, and a remote control for controlling at least one of the insulin pump and the analyte sensor, the method comprising:
providing the at least one unit and the at least other unit;
determining whether a first frequency is available for transmission of a message related to operations of the dispensing system, wherein said determining includes performing a comparison of detected radio frequency interference (RFI) associated with the first frequency to an associated RFI threshold; and
determining a second frequency to transmit the message in response to the determination that the first frequency cannot be used to transmit the message.
